(19) United States
(12) Patent Application Publication
(10) Pub. No.: US 2022/0151541 A1
(43) Pub. Date: May 19, 2022

(54) Computerised and Automated System for Detecting an Allergic Reaction and Managing Allergy Treatment

(71) Applicant: Dosepack LLC, San Juan, PR (US)

(72) Inventor: Miteshkumar Ishwarbhai Patel, San Leandro, CA (US)

(21) Appl. No.: 17/098,548

(22) Filed: Nov. 16, 2020

(51) Int. Cl. A61B 5/00; A61B 5/01; G16H 20/00

(52) U.S. Cl. CPC A61B 5/411; A61B 5/01; A61B 5/0077; A61B 2562/0271; A61B 5/7475; G16H 20/00; A61B 5/0008

(57) ABSTRACT

A computer implemented and automated method for detecting and measuring an allergic reaction is described. The method utilizes a server computer, an infrared module, a camera, and electronic devices connected through a network to measure allergic reactions as well as manage a patient's end-to-end allergic treatment.

The infrared (IR) module includes a temperature sensor to detect and read radiation and provide a temperature reading emitted from the wheal formed on patient's body in response to an allergen.

The camera captures an image of the wheal and provides its dimensions.

IR module and camera correlate data to verify accuracy and provide criticality of the reaction.

The method also provides other modules such as vaccination module, scoring module, allergen recommendation module, and a skin test result management module. All modules are provided on an electronic device and allow user input and changes to manage allergy treatment.

### FIELD OF THE INVENTION

The present invention generally relates to a computerized and automated system for detecting an allergic reaction and managing allergy treatment of a patient. More specifically, the present invention relates to a system that uses an infrared device, a camera, a server, and plurality of connected devices to measure and quantify a reaction on a patient's body when tested (or injected) with an allergen. The present invention also relates to a system for determining phenotypes for an allergen. The present invention further relates to a system that records all allergy treatments and provides treatment updates, scheduling, and billing for the treatments provided. Additionally, the present invention also relates to a system that provides a user interface for its operator to review and change parameters and modify system features, including providing input on vaccines and allergen mixtures and concentrations.

### DISCUSSION OF THE RELATED ART

Numerous adults and children suffer from one or another type of allergy. In many cases, the allergy results in teary eyes, runny nose, nasal congestion, sneezing. In some severe cases, the allergy may be life threatening that causes breathing issues and other severe vital sign fluctuations.

Some allergies are seasonal, such as caused due to pollen, spring time weather and others are year-round. It is not always seasons changing, it can be other allergies that show up in on our skin as well as in our digestive and respiratory systems.

The first and foremost step in the treatment or containment of allergy is to determine the root cause and what triggers the allergy. To determine this, a variety of tests are performed on the person. One such common test is known as the skin prick test.

The skin test, which may also be known by other names such as prick text or puncture or scratch test is performed to determine and identify the type of allergy from which a person suffers. Allergist, a type of doctor that specializes in allergy treatment, typically administers or prescribes the allergy skin test on patient to determine the substances that cause the allergy and uses the results to diagnose the patient and present containment options.

The test can consist of various panels of allergens being injected or placed on the person's skin. Anywhere from a small quantity up to a panel of 80 allergens can be used at a time to test the patient's response to various allergens. In some severe cases, more than one panel may also be used. The panels may be further subdivided into specific panels of allergens, such as types of grass, foods, animals, plants and more.

The test is administered by a skilled technician, or a nurse, who places pricks the person's skin with tiny needles. These needles are called lancets. Other names have also been used in the past. Using these needles, a small amount of allergen is injected or introduced into the person's skin. The test is typically administered in the clinic, doctor's office or a monitored facility. The reason is that some people may have a severe reaction, could also be an anaphylaxis reaction that may be deadly, and as such needs to be performed in a controlled and monitored environment where a doctor is present or easily accessible.

The skin reacts to these allergens, sometimes right away and sometimes after a duration of time.

In more scientific terms, the reactions are the result of an overreaction of the person's immune system when it is irritated by the allergen. When faced with an irritant, the immune system releases immunoglobulin (IgE) antibodies—this is the body's way of trying to fight off the irritant, i.e. the injected allergen. The skin test is performed to detect these IgE antibodies and quantify them.

In an attempt to quantify them, the reactions are measured after the skin has had time to react. The measurement is basically the measuring of the inflammation, the redness, or rash that extends in a circular (sometime other shapes) fashion outside the area of the allergen. A swelling or itch around the area of the allergen is common. Typically known as a wheal (or wheel)—analogous to a wheel of a vehicle, the reaction is a circular reaction. The wheals, which were provoked by the allergens, and are considered positive above certain cut-off diameters. Typically, the allergy industry considered anything beyond a 3 mm (and sometimes 2 mm) diameter to be a positive reaction.

The technicians measure the reaction using a ruler. As mentioned earlier, the wheal may not be exactly circular and the reaction may take another shape, the measurement of diameter from the center is based on both skill, human judgement, and some guess work that albeit leads to a measurement but which may imprecise.

Some advances in the measurement methodology have resulted in companies producing better measuring techniques using electronic rulers, and other hand held devices. Some have also taken measurements by hand in all directions, fed them into an algorithm or an excel sheet to provide the average deviation and come up with a approximate measurement.

Some advances in this space, which are still limiting, includes systems for providing and tracking immunotherapy treatment. For example, one such immunotherapy tracking treatment system is described in U.S. patent application Ser. No. 14/775,706, titled “Immunotherapy system and method thereof” (herein after referred to as the “Daines patent”).

The Daines patent focuses on narrowing down all tested allergens to the 5 most likely allergens to be causing the allergy symptoms and generating patient specific immunotherapy treatment recommendation. It does so by using a software platform for entering patient allergy results, including patient medical history. The system uses a processor to generate an immunotherapy treatment recommendation from the inputs provided by its user. Subsequently, the system includes a step to verify the immunotherapy treatment recommendation.

In addressing the goal on how to narrow down the set of allergens to a set of 5 allergens, the Daines patent determines the concentration mixtures of Allergens, sometimes tests 50 or more allergens, to then narrow them down to a set of 5 allergens. At various times in the patent, the Daines patent reiterates its focus of “treating for fewer allergens is more efficacious than treating for everything the patient is allergic to.”

With respect to providing recommendations, the Daines patent provides “a treatment recommendation with one or more allergen to be administered to a patient over a predetermined time, e.g., 1 allergen, 2 allergens, 3 allergens, 4 allergens, 5 allergens, 6 allergens, 7 allergens, 8 allergens, 9 allergens, 10 allergens or more. More preferably, 5 allergens make up the treatment recommendation”. This recommendation is to further the goal of the patent to narrow the set of a large group of recommendations down to a group of 5 allergens. [Daines patent Paragraph 53]

The Daines patent elaborates that its “allergy treatment recommendation module is configured to generate a specific treatment by programmatically deducing an immunotherapy treatment recommendation based on one or more of the patient's history, science factors, and results from the allergy test. The immunotherapy treatment recommendation includes one or more allergens configured to elicit an immune response”.

Although the Daines patent attempts to apply advances in computer and software systems to allergy, it does so for mostly narrowing down the set of allergens and providing a recommendation based on the patient's past history. It does not, however, provide tools for measuring a reaction, correlating the reactions with other factors, analyzing the measured reaction, and then considering the patient's overall characteristics in providing a treatment option. The treatment option suggested is mostly focused on providing an allergen and not on containing the reaction or providing remedy to the allergy.

Yet another attempt to manage allergy treatment is described in U.S. patent application Ser. No. 15/379,276, titled “Allergy Service Management Portal” (herein after referred to as the “Smith patent”).

The patent focuses on receiving allergy results and scoring them. It does so by receiving a skin wheal image captured by the imaging device, and identifying an antigen based on the skin wheal image and the patient-related information. The patient related information is obtained by interfacing a physician's computer terminal. The patent scoring and ranking is based on an allergenicity score and a seasonality of the antigens, among other things. Once the scoring and ranking is completed, the patent calculates a prescription recommendation based on the determined environmental antigen and provides the calculated prescription recommendation to the user's device. Again, the patent has a narrow focus of scoring and then converting the results into a prescription. It does not, however, provide a complete management for an allergy patient and does not perform the start to end steps of measuring a reaction, correlating the reactions with other factors, analyzing the measured reaction, and then considering the patient's overall characteristics in providing a treatment option.

As such there is a need for a better solution that provides accurate measurements of the wheal in measuring allergic reactions, analyses the reactions, and manages the treatment of the patient's allergy.

### SUMMARY OF THE INVENTION

A computer implemented and automated system for detecting an allergic reaction and managing allergy treatment is disclosed. The system includes a plurality of electronic devices communicatively connected to each other and centrally to a server. The devices include an infrared module, a camera, a server computer, and a plurality of computing and mobile devices connected wirelessly to the server.

The server computer includes a plurality of components that are interconnected to each other either through digital circuitry or wireless means. The components include one or more processors, a bus for communications, server memory for storage of data, a database, an I/O device and a network interface.

In one embodiment, the system is used in relation to a skin test performed on a patient. The system is used before the skin test for planning purposes and during the skin test. Planning use includes the processor of the server causing a user interface to appear on one or more connected computing and mobile devices and allowing a user to operate the system as well as modify a plurality of features or provide input into the allergy skin test as well as allergy treatment management of a patient. Using the user interface, the user/operator can create a digital footprint of the allergens that will be injected into the patient in a grid-like pattern. The digital footprint in the user interface represents the actual allergens on the patient and the user can use the interface for various planning purposes. One such planning purpose it to group related allergens into a panel.

The system is used during the skin test to electronically record placement of allergens in a grid-like pattern on the patient's body, either separate allergens or allergens grouped in panels. It is also used to detect any reactions in response to the allergens and mark the reactions in the digital footprint.

The reactions of the skin test are measured by the infrared module and the camera. In operation, the server computer accesses the infrared electronic module which includes a body, a handle, an infrared temperature sensor connected to a microprocessor, and a trigger button to operate the infrared electronic module.

The server computer then activates the infrared electronic module. Such activation results in the infrared electronic module's infrared temperature sensor sensing the heat around the allergen and obtaining a temperature reading of a wheal formed on the patient's body. Wheals of different sizes are formed as a type of reaction in response to an allergen tested or injected into the patient's body.

The infrared electronic module is also connected communicatively to the camera or image scanning module. They both work with each other to determine the temperature and verify the wheal image as a first and second point of verification. The system also uses the camera and the IR Module as a secondary check to verify each other's data. It does so by comparing data obtained by the camera with data obtained by the IR module, or vice versa, and runs a logical computation to determine they are within a predetermined range of possibilities and if not, concludes that one of the devices, either the IR module or the camera, has a reading error and performs the readings over again.

Since the server computer and the infrared electronic module is communicatively connected, which can be through wireless means, the infrared electronic module transmits the temperature reading of the wheal to the server which is then stored in the server memory.

Similar to the interactions between server computer and infrared electronic module, the server computer also accesses the camera and activates it to take an image of the wheal. The camera responds by focusing its lens on the wheal and capturing the image of the whole wheal along with its dimensions. The camera then send the captured image and dimensions to the server which are then stored in server memory.

The processor of the server computer analyses all the data received, i.e., image and dimensions as well as temperature reading. Since there needs to be a correlation between the wheal dimensions and temperature reading, the processor analyses both readings to determine if an accurate reading was obtained. In the event of an error, the system either alerts the operator to take another reading or automatically takes a second reading and subsequent readings to resolve any inaccuracies.

The system also performs other type of analyses, such as scoring each allergen in relation to the readings obtained and determining if a reaction has occurred. The results of the scoring as well as any reactions and comments are displayed on the user interface.

The system also includes an image module that performs image processing. The image processing includes examining the clarity and completeness of the image and determining if the dimensions of the wheal were captured accurately. In the event of errors or pixelated images, the system tries to enhance the image through corrective measures and alternatively required a second image to be scanned.

The camera as mentioned above can be of a few different types. In one instance the camera is a handheld portable device that can be used by the operator to manually hover over the wheal and capture the image. In another instance the camera can include a grid-patterned body, similar to the grid that is created on the back of the patient's body for injecting allergens, and each cell of the grid includes a camera having a lens that can individually take an image of one specific wheal and together the camera body is equipped with enough cameras to take the image of the entire back of the patient's body and capture all the allergens injected and any wheals associated with them. In yet another embodiment the cameras include flexible and bendable probes that follow the contours of the body to capture the images of a wheal.

During the skin test, the system is also capable or forward prediction analysis. Machine learning principles and algorithms can be applied to compute these forward predictions. For example, machine learning can continually monitor test as well as use data from previously tested samples to enhance forward predictions accuracy and be able to determine a reaction faster and more accurately before it occurs.

Using forward prediction analysis, in the event that an allergen being tested on a patient is leading to a reaction that can be critical or cause an emergency, the system is capable of calculating such a scenario before it occurs and providing an alert in real-time such that the provider or medical doctor can adhere to the emergency before it occurs. The system also causes the testing to automatically stop if such an occurrence is predicted.

In addition to its usage for planning a skin test and managing all aspects of the skin test, the system is also used for multiple other purposes. Yet another purpose includes determining allergen specificity. In this embodiment, the system navigates through complex decision and logic trees to determine allergen specificity which leads to a better allergen being used in testing and a better-informed diagnosis.

The system is also used for recommending antigens and refining concentrations in building vaccine vials. In so doing, the system provides a user interface such that the user may provide their input on the level and types of mixtures and concentrations to be used in constructing a vaccination. The system also provides alerts if any two antigens cannot be mixed.

The system is also used to analyze phenotypes for an allergen. The system automatically analyses the phenotypes based or a positive of negative reaction of the allergen.

The system also serves as a complete record of the patient history and treatment relating to allergy management. For example, the system automatically creates visit notes and generates billings and invoices. Further, the system also provides scheduling, calendaring, and reminders to the users that can be set using the user interface provided.

### BRIEF DESCRIPTION OF THE DRAWINGS

The accompanying drawings are included to provide further understanding of the invention and constitute a part of the specification. The drawings listed below illustrate embodiments of the invention and, together with the description, serve to explain the principles of the invention, as disclosed by the claims and their equivalents.

FIG. 1 depicts one embodiment of a system that is used in conjunction with the testing allergic reactions and managing allergy treatment according to the disclosed embodiments;

FIG. 2 is an example of a computer system used for performing the steps of allergy detection and treatment according to the disclosed embodiments;

FIG. 3 illustrates a plurality of allergens placed in a grid on the backside of person during a skin test according to the disclosed embodiments;

FIG. 4 illustrates an infrared module used for detecting and reading an allergic reaction according to the disclosed embodiments;

FIG. 5 illustrates the internal components of the infrared module and its communication with the server according to the disclosed embodiments;

FIG. 6A illustrates the back of a patient's body with a plurality of wheals that occur as a reaction to the allergens injected according to the disclosed embodiments;

FIG. 6B illustrates the single wheal and the density of a reaction that has occurred on the patient's body in response to an allergen injected according to the disclosed embodiments;

FIG. 6C illustrates an exemplary detail snapshot of a wheal that includes measurements, scores, and temperatures according to the disclosed embodiments;

FIG. 6D illustrates a temperature range and its associated reaction as calculated by the system according to the disclosed embodiments;

FIG. 7 illustrates a handheld camera used for capturing an image of a wheal according to the disclosed embodiments;

FIG. 8A illustrates an automated and secured camera used for capturing an image of a wheal according to the disclosed embodiments;

FIG. 8B illustrates a detailed cross-section of the camera of FIG. 8a and its lens according to the disclosed embodiments;

FIG. 8C illustrates a detailed cross-section of the camera of FIG. 8a and a selected panel of lenses according to the disclosed embodiments;

FIG. 9A illustrates an automated and secured camera having a plurality if movable prongs placed on top of the body to capture the image of a wheal according to the disclosed embodiments;

FIG. 9B illustrates a detailed view of an automated and secured camera having a plurality if movable prongs according to the disclosed embodiments;

FIG. 10 illustrates a block diagram depicting one exemplary process of reading and analyzing a wheal formed due to an allergic reaction according to the disclosed embodiments;

FIG. 11 illustrates an architectural block diagram depicting the system used by the IR module and camera according to the disclosed embodiments;

FIG. 12 illustrates a block diagram depicting the central system module and its communications and connections with a plurality of physical and computational objects that are part of the system according to the disclosed embodiments;

FIG. 13 illustrates a block diagram depicting one exemplary process of the central system module determining a phenotype for an allergen and displaying test results according to the disclosed embodiments;

FIG. 14 illustrates a block diagram depicting one start-to-finish cycle of performing a skin test and updating an electronic health record according to the disclosed embodiments;

FIG. 15 illustrates a detailed submodule A (also referred to as Module A) that is used as part of the exemplary start-to-finish cycle described in FIG. 14 according to the disclosed embodiments;

FIG. 16 illustrates a detailed submodule B (also referred to as Module B) that is used as part of the exemplary start-to-finish cycle described in FIG. 14 according to the disclosed embodiments;

FIG. 17 depicts on exemplary scoring of allergens as displayed on a user interface that is communicatively coupled to the central system module according to the disclosed embodiments;

FIG. 18 illustrates a detailed submodule C (also referred to as Module C) that is used as part of the exemplary start-to-finish cycle described in FIG. 14 according to the disclosed embodiments;

FIG. 19 illustrates a detailed submodule D (also referred to as Module D) that is used as part of the exemplary start-to-finish cycle described in FIG. 14 according to the disclosed embodiments;

FIG. 20 depicts on exemplary screen of the user interface that shows a set of exemplary parameters for updating the skin test according to the disclosed embodiments;

FIG. 21 depicts one exemplary screen of the user interface that shows wheal flare value determination according to the disclosed embodiments;

FIG. 22 depicts one exemplary screen of the user interface that shows skin test order and its parameters according to the disclosed embodiments;

FIG. 23 depicts one exemplary screen of the user interface that shows concentration columns for an intradermal test according to the disclosed embodiments;

FIG. 24 depicts one exemplary screen of the user interface that shows a skin panel creation according to the disclosed embodiments;

FIG. 25 depicts one exemplary screen of the user interface that shows a numeric keypad for entering wheal and flare results according to the disclosed embodiments; and

FIG. 26 illustrates a block diagram depicting one start-to-finish cycle of performing a skin test and managing end-to-end allergy treatment, scheduling, and billing according to the disclosed embodiments.

### DETAILED DESCRIPTION

Although the specification concludes with claims which particularly point out and distinctly claim the invention, it is believed the present invention will be better understood from the following description.

The present invention can comprise, consist of, or consist essentially of the elements that are described in the specification. However, the invention is not so limited and any of the additional or optional ingredients, components, or limitations described herein are contemplated within.

The citations of any document are not to be construed as an admission that it is prior art with respect to the present invention. For example, certain patents have been cited in the background that are merely for exemplary purpose and not to be construed as prior art.

In this application, the term skin, when used is to be construed as an outer covering of a human being that consists of two layers of cells, a thick inner layer, also known as dermis, and a thin outer layer, also known as the epidermis. The epidermis is the external, nonvascular layer of the skin in which a prick is inserted for the purposes of an allergy skin test.

In one embodiment of the present invention, a method of conducting a skin allergy test using a prick or skin test is described. The data resulted from the skin test is further examined through sophisticated computer logic having a processor navigate various logic decision trees to both analyses the obtained data as well as correlate the data with various elements both related to the patient and external to the patient. The treatment of the allergies that were discovered through the skin test is provided by the processor accessing a plurality of resources providing a customized treatment plan. The treatment plan is then managed by an automated computer system. Various features of the system along with the methods used for performing an allergy skin test, reading and analyzing the skin test, and the post processing the results for treatment and management are described.

As such, various aspects of the invention are disclosed in the accompanying description and alternative embodiments of the present invention and their equivalents are devised without parting from the spirit or scope of the present invention.

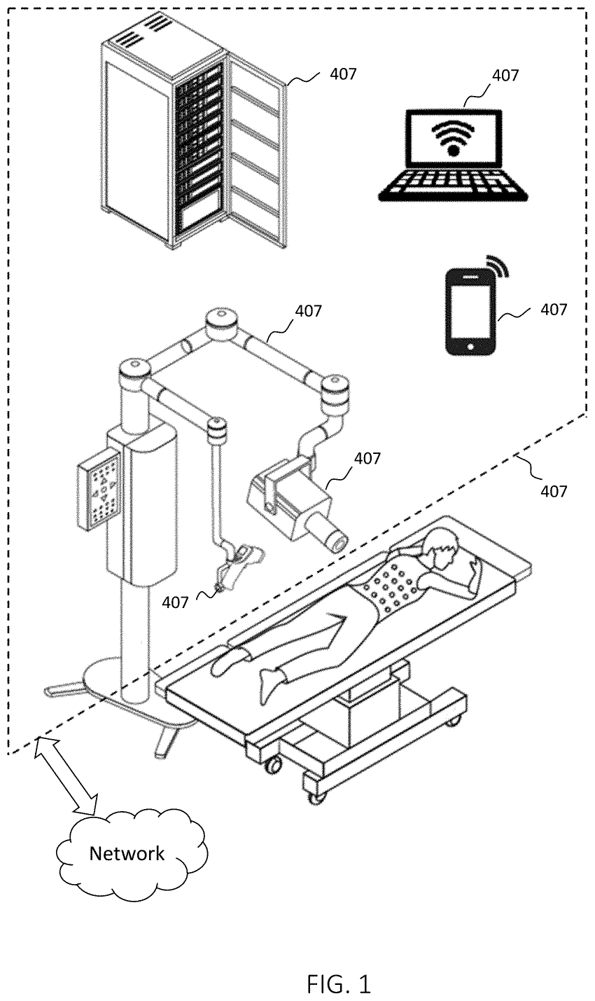

FIG. 1 depicts one embodiment of a system that is used in conjunction with the testing allergic reactions and managing allergy treatment according to the disclosed embodiments.

Embodiments of the system 100 provide an inventive apparatus(es), computer-based data processing algorithms and methods for conducting a skin allergy test, obtaining and using the results from the conducted test—i.e. data relating to allergic reactions, and management of allergy.

In one embodiment, the system includes an allergy evaluator electronic device and a computer module. The IR Module and the computer module can be separate modules, or, in another embodiment they can be integrated in the same module and enclosure. Alternatively, the IR Module and the computer module can communicate with each other via a wireless connection, such as Bluetooth or other wireless connection.

In yet another embodiment the IR Module can be used for telemetry and for transmitting skin allergy data to a remote location, e.g., via a telephone line, Internet, and/or wireless phone to a caregiver, physician, or dermatologist.

The system 100 includes a server 103, a computing device 105, a camera 107, and an infrared module 109. The server computer 103 includes its own processor, server memory, and server network interface.

The system 100 is communicatively connected to a network 111. The systems are used during an allergy skin test performed on a patient 113 to read the allergic response or allergic reaction on the patient's skin due to a plurality of allergens 115 placed on the patient.

The computing device 105 may be web-enabled device that include a browser application configured to receive and to send web pages, web-based messages and the like. The browser application may be configured to receive and display graphic, text, multimedia, or the like, employing virtually any web-based language, including a wireless application protocol messages (WAP), or the like. It may also include at least one other client application that is configured to receive content from another computing device, including, without limit, the server 103. The client application may include a capability to provide and receive textual content, multimedia information, or the like. The client application may further provide information that identifies itself, including a type, capability, name, or the like.

The server computing device 103 may be part of a clinic, and allergist office, or a medical facility where an allergy test is conducted or where a patient with allergy visits to see a medical professional.

A network 111, such as a local area networks (LAN), wide area network (WAN), or a wireless network shown as network may be communicatively connected to the system 100 such that components outside of system 100 can communicate with the system to send and receive information, such as allergy related information or patient related information.

Other devices not shown, such as electronic portable devices (e.g. cellular telephones, smart phones, radio frequency-enabled devices, personal digital assistants, handheld computers, tablets, laptop computers, wearable computers) may also be communicatively coupled to the system 100.

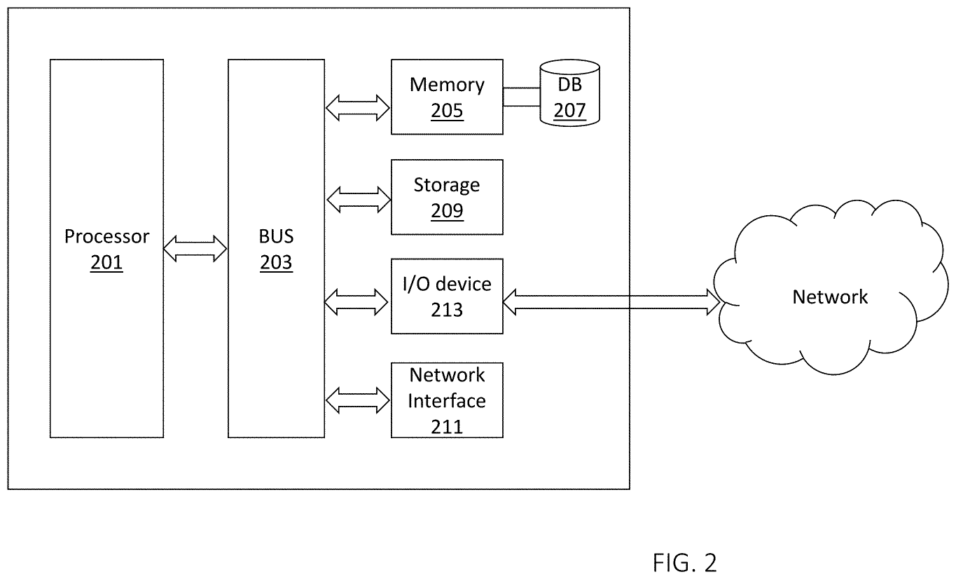

The server includes processor, memory and other electronic components that are described in further detail in FIG. 2. The system 100 allows communications between components within the system through wireless means such that all information can be shared with the server and also between components as guided by the server.

The infrared module 109, also referred to as the IR Module, is part of the system 100. The user interface displayed on the screen of the IR Module or other suitable handheld/portable device displays allergy information and also allows the operator of the IR module various features and functionalities.

When the IR Module is in its real-time display mode, it reads the level and type of reaction as you scroll over a patient's body in the areas that have been pricked with allergens for the skin test. As you scroll over a reaction, the system has the capability of generating an alarm that is indicative of the reaction. For example the alarm can be a sound, such as a beep, a vibration, a light, or other visual or audible alert to inform the operator that a reaction has been detected. The severity of the reaction can also be captured with different types or alarms.

The IR module also shows the temperature readings in numerical and graphical representation of the wheal that is formed as a reaction to the allergen tested. The device includes features such as zoom and scroll functions on the touch screen. The user can use two fingers pinch and expand touches on the screen to zoom in and out of a specific area of the patient's body.

System's Camera 107 can be an individual handheld portable unit or mounted on a fixed structure, such as a wall or stand. In its handled mode, an operator can maneuver the camera and hover it over the patient's allergens to obtain a visual representation of the allergic reaction. When used as such, the camera is capable of taking an image of the wheal, i.e. the area that reacts to an allergen, and able to capture the data for image processing.

In its mounted mode, the camera may be attached or be part of a mechanically moving structures 106, such as an arm, lever, or other mechanically moving part 106. The mechanically moving structure 106 may be attached to a wall or a stand or be part of another machine or structure. The mechanically moving structure 106 can be moved by the operator such that the camera 107 attached hovers over the patient's 113 back and above the allergens 115. The process may also be automated such that mechanically moving structure 106 detects the patient 113 on its own and moves to allow the camera 107 to be above the allergens 115.

The camera 107 and the IR module 109 communicate with each other through the server. Alternatively, they may communicate with each other directly. The camera 107 and the IR module 109 may communicate to exchange allergy related information or to guide one another through the allergy skin test process.

For example, in one embodiment, if the IR module 109 detects that a reaction has taken place, it may communicate with the camera through the server, thereby resulting in the camera being moved above the allergens to capture the image of the reaction cased by the allergen. Other types of communications between the camera 107 and the IR module 109, such as exchange of allergy information or signaling or scheduling for next steps, are also contemplated.

FIG. 2 is an example of a computer system used for performing the steps of allergy detection and treatment according to the disclosed embodiments.

In one embodiment, the computer 200 includes a processor 201. The processors communicatively connect via a bus 203 to a memory 205, which is connected to the database 207.

The processor is also communicatively connected to a secondary storage 209, and an input/output interface 211, and a network interface 213, through the same bus 203. The computer operates with any known operating system, such as Microsoft Windows, MacOS, iOS, Android, or Linux.

The memory 205's database 207 contains allergy related information for a plurality of patients. A variety of analyses may be performed by using the information in the database 205 with outside resources and skin tests performed or other internal information. For example, an older test result stored in the database may be used to compare with a newer test performed and analyzed to review patient's progress. The results may be recorded in memory.

The memory 205 can include random access memory (RAM), read only memory, flash memory, or any other suitable memory systems. The memory module may store patient, medication, and allergy, allergens, or other patient's medically related data.

The network interface 211 may transmit messages from the computer 200 to other computers or mobile devices as well as receive messages and instructions form such devices. The input/output interface 213 may include, touch screen, keyboard, or a display unit.

The computer system uses software program(s) that are configured to operate on a processor, such as a standard microprocessor. The processor functions to process instructions and navigate decision logic to made decisions, such as to determine the extent of an allergic reaction, and report the results. The processor also processes images obtained by the camera as well as the IR module.

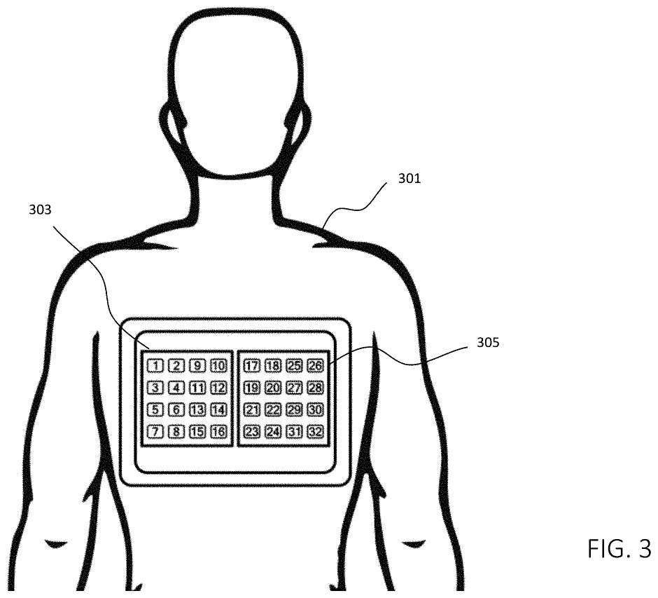

FIG. 3 illustrates a plurality of allergens placed in a grid on the backside of person during a skin test according to the disclosed embodiments. A skin allergy test is performed to determine if the patient is allergic, and if so to which allergens and that severances of each such allergy. The skin test is performed by a trained nurse or an allergy specialist or even the allergist/doctor.

Since allergens are discussed throughout the application, we provide a general definition and understanding here. Basically, allergens are substances that can cause an allergic reaction. Although there are several types of allergens, typically the main allergens can be categorized into three primary types. These include allergens that can be inhaled—i.e. airborne, allergens that are in your food, and allergens that we come in contact with—such as grass or plants or poison ivy etc.

For airborne allergens, they affect the body when they come in contact with the lungs or membranes of the nostrils. One common example is pollen. Typically, people that have hay fever or allergic to pollen will have a tougher time or shown a stringer reaction is spring or on days when the pollen count is high.

Food allergens can be nuts, gluten, peanuts, or any other foods that may be intolerable to a specific person. Some are lactose intolerant and some have an allergy to shellfish—it varies from person to person.

Although there are multiple ways to test for allergy, such as blood test or a skin test and in cases of food allergies, an elimination diet or some restriction from certain foods, we will focus here on skin test and how it is performed.

When a skin tests is performed, it is used to determine which allergen is causing the allergy to the patient being tested. Since at the outset it is now know which allergens can cause the allergy to the patient, or at least not fully concluded that the known allergens are the only contributing factor, a trial and error approach or a most calculated approach to test, use the results as a feedback, and retest types of methodologies can be applied in such testing.

In some cases, before a skin test, another test, which is similar called the scratch test may also be performed by the trained professional, e.g. a trained nurse, an allergy specialist, or allergist/doctor. The essence scratch test is to determine or narrow down an allergy to either prevent a skin test or to narrow down some allergens that can be used in a more narrowly focused skin test. The scratch test is basically a smaller version of the skin test.

The scratch test consists of placing a liquid on a portion of the patient's skin. Typically, a tool is used for placing the liquid and then the same tool is used to lightly puncture the allergen into the skin's surface to determine if a reaction occurs. Based on the results of the scratch rest, or lack thereof, the next step is to perform a skin test. In some instances, the scratch test is bypassed and the medical professional may go directly to the skin test.

As shown in FIG. 3, when performing a skin test, the skilled medical professional will mark the back side of a person's body 301 where skin test is to be performed. The marking is show as 303 and 305.

In 303, also referred to as a panel of allergens, the skilled medical professional may have used a plurality of allergens that are related to a genre. For example, the skilled medical professional could have used all airborne allergens in this panel. To distinguish it from another panel, typically they are grouped together and in some cases a box is drawn around them to delineate them from other allergens. Alternatively, a box may not be drawn around them.

Likewise, in the 305 panel, the skilled medical professional may have used a plurality of allergens that are food related. These could include allergens relating to commonly household, such as cat hair, carpet fiber, types of dust, common grass that is grown in backyards etc.

The skilled medical professional may also combine one or more allergens to develop an allergen mix or compound. At times mixture of one food with another food may release a certain reaction which may not have occurred when the food is taken separately. Likewise, when one drug is combined with another drug or with a particular food it may result in a certain reaction which may not have occurred when the drugs or food were ingested separately at different times. As such, certain mixtures may be combined to test the composite allergen mixture on a patient as well.

Again, to distinguish the common household allergens from panel 303, the skilled medical professional may have grouped them together and drawn a box around it.

Panel 303 and 305 panel are just some exemplary panels. Other panels that include various type of pollen, foods, materials specific to a region, season specific allergens are among those also contemplated. Regardless of the type, they are injected into the patient's body and used to determine a reaction.

The allergens that are used in the skin test are specially manufactured and not the actual substance in its raw form—e.g. to test for an allergy to grass, actual grass is not injected into the patient, rather a specially manufactured form that represents grass is used. These specially manufactured allergens are aqueous solutions of proteins extracted from the relevant materials. They are then combined with 50% glycerol which acts as a preservative. The allergens are viscous and supplied in bottle that can be used in the form of droplets that can be injection into the patient's body.

Once the back of the patient's body is marked up, the skilled medical professional uses these bottles to inject the allergens into the specific marked areas of the patient's body and then mark that such allergens were injected into the system computer. The software program that is used to enter the information communicates the entered information with the processor and stores it into the memory and database as show in FIGS. 1 and 2. Alternatively, the system may use a tool that is wirelessly connected to the processor and every time an allergen is injected, it is automatically recorded into the system.

In another embodiment, the system may provide step-by-step guidance to skilled medical professional that may be new to the process. The skilled medical professional can select the level of guidance needed, e.g. a lesser level of guidance or options to bypass certain features for a medical professional with higher skill and step-by-step guidance for someone that is new to the process or the system. If step-by-step guidance is selected, the system guides the medical profession on which allergens to obtain and exactly where to inject them into the specifically marked areas of the patient's body. In one instance, the step-by-step guidance is displayed on a user interface and can be made interactive such that an operator can scroll through the steps displayed and ask for further assistance or clarification as needed.

Further, the system may guide them after injecting is completed. It may mark the allergens as injected into the software and start a timer for the amount of time the allergens are to be tested and observed. The timing may be adjusted based on patient's response or if the system detects through its forward predictions analysis that a reaction is likely to occur.

The skilled medical professional allows the allergens to takes its course and waits for a certain period of time to determine if a rection occurs. Once a reaction occurs, the skilled medical professional records the wheal and flare of the reaction that signifies the extent of the reaction. Alternatively, the reaction may also be measured and recorded through use of the camera and the IR module as depicted in FIG. 1. Both camera and the IR module are described next in further detail.

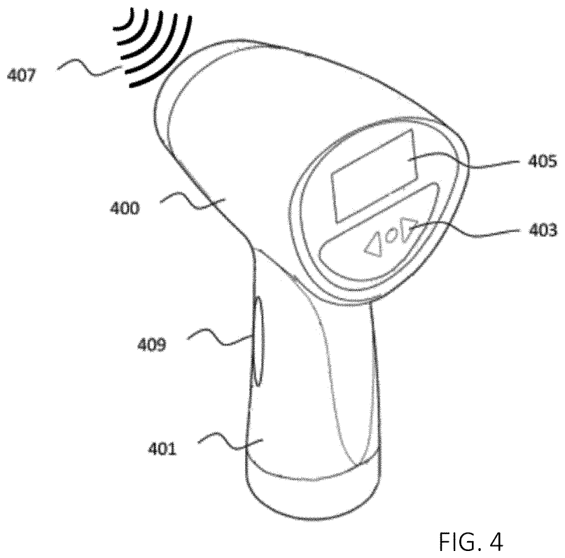

FIG. 4 illustrates an infrared module used for detecting and reading an allergic reaction according to the disclosed embodiments. As shown in FIG. 4, it depicts a handheld infrared module 400, also referred to as IR module 400, the infrared skin allergy detector, or skin allergy detector, or infrared gun. The IR module includes a plurality of features that aid the user in detecting an allergic reaction on a person's body as well as measuring the allergic reaction.

The IR module 400 has a handle 401 and a plurality of operational buttons 403. The buttons allow an operator to access and activate features of the IR Module 400. The IR Module 400 also includes a display 405.

The operation of the buttons 403 activates a processor to access and execute instructions that reside in a software module that resides within the IR Module 400. The buttons can be of any size and shape and can vary in materials. Alternatively, the display may be a touch screen display overcoming the need for any physical buttons to activate or execute the features of the IR Module.

In operations, the operator points the IR Module 400 in the location of the area where the allergens are injected. There may be guidance provided by the IR Module to accurately point the IR Module in the right areas.

For example, in one embodiment, the IR module 400 projects a light is used for guiding purposes to aim the IR module 400 at the right area of the patient's body. In this embodiment, a light is projected from a small aperture on the IR Module 400 that houses the lens. The light can be a normal light or a laser light may be projected outward and aimed at the area of testing thereby guiding the operator of the IR Module to point it accurately over the allergen area.

In another embodiment, the IR Module 400 may allow the operator to use their own experience in aiming it at the testing area and capturing the IR energy that emanates from the testing area.

In either case, the IR module 400 is aimed at the wheal area to read and analyze a reaction that is generated in response to the allergen injected. The IR Module 400 also includes an internal component, the optical lens that will focus on the IR energy which is coming out from wheal. The optical lens will guide the IR energy on the IR module 400. After processing the final value will give the temperature readings. The temperature readings will show on display 405 of the IR module 400.

In response to the activation of the IR Module 400, when aimed at the patient's body in the area of the allergen injected, the body radiates and the radiation comes from the skin test area and the data of the radiation is captured by the lens of the IR module 400.

The IR Module also includes an infrared temperature sensor connected to a microprocessor. The infrared temperature sensor performed the temperature measurements and reading of the wheal that was formed in response as an allergic reaction to an allergen injected into the patient's body.

IR module 400 also includes internal components that are not shown in FIG. 4. These features include a rechargeable battery. The battery can be a standard lithium-iron battery or other batteries that are rechargeable and hold the charge for the duration of a skin test or longer.

The IR module 400 has a trigger 409 that can be pressed by the user to activate the IR module 400 and have it activated such that the IR Module is capable of reading the radiation and data that is emanated from the reaction area. The activation button is part of the main body of the IR module 400 and attached to its handle.

Internal to the IR module 400, a processor, memory, and a lens are included. Additionally, a means for measuring is also communicatively coupled to the processors and the memory. The battery provides the measurement module and the internal hardware the power it needs to perform its intended function, i.e. detect an allergic reaction and perform a measurement.

The display 405 can be an LCD panel touch screen or a simple display screen that does not have touch screen features. In the event of it being a touch screen, a user can operate the IR module 400 using the different modes of operation as presented on the touch screen, including performing a measurement and having the measured allergic details depicted on the touch screen 405. The software downloaded on the IR module 400 allows the touch screen features to be navigated by the user.

To measure a testing area, i.e., a particular allergen or a panel of allergens, the user can aim the measurement mouth—i.e. the area shown in FIG. 4 adjacent to the IR rays, and point it directly at the target. By pulling the trigger 409, the user allows the IR module 400 to get the results of the temperature reading based on the IR energy obtained from the wheal or testing area.

When the user selects the various software functions from the touch screen or presses the trigger 409, a measurement is made. The processor processes the measurement and stores the results in the memory.

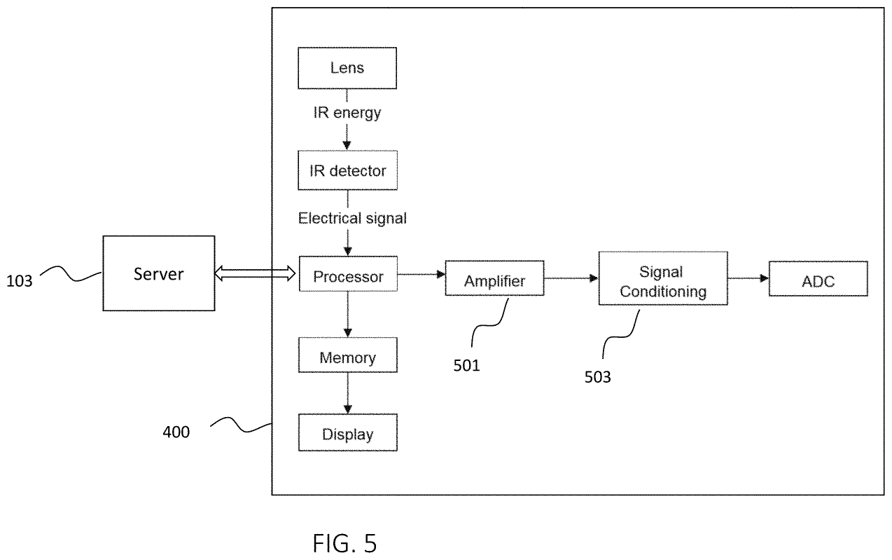

The IR module 400 communicates with other components in the system as shown in FIG. 1. Additional communications are shown in FIG. 5. The communication of the processor with the measurement mechanism is for the purposes of processing the allergic reaction. At times when the reading cannot be obtained, the processor may also provide a visual or voice alert to the user.

The IR module 400 can also program the IR module 400 such that it can automatically take measurement and record them as the user hovers over the various allergens or panels of allergens as shown in FIG. 3. Using such preprogramming, the user can also run extensive tests and measurements to verify the reactions. All such data is then provided to the processor for analysis and then storage in the memory.

The user may also activate the measurement mechanism by using the trigger 409 resulting in a heat generation from the IR module 400 that is focused on a wheal generated due to the reaction of an allergen. AS mentioned earlier, the body emanates radiation and that is captured by the optical lens that will focus on the IR energy which is coming out from wheal.

One method to stop the testing or stop the IR module 400 is by releasing the trigger or alternatively pressing a stop button on the touch screen. The release of the trigger results in stopping the IR module 400 from performing measurements. If the detected temperature exceeds the preset limit, the IR module 400 will generate audible alarm. Alternatively, in a preprogrammed mode, the IR module 400 will automatically stop once the user has hovered over all the allergens or panels of allergens or wheals.

FIG. 5 illustrates the internal components of the infrared module and its communication with the server according to the disclosed embodiments. The internal components of IR module 400, as mentioned in description of FIG. 4, include optical lens, IR detector, processor, memory, amplifier, signal conditioning circuit 503, ADC, and display.

The amplifier 501 amplifies the signal such that a better reading can be obtained.

The conditioning circuit 503 is a mechanism that is designed to improve or enhance the signal quality such that a better allergy reading can be performed. In operation, the signal conditioning circuit 503 uses magnetic isolation which then transforms the signal from a voltage to a magnetic field such that the transformed signal can be transmitted without physical connection and through signal rays, electronic signal, or similar means. Basically, the signal is modulated to enhance transmission. Specifically, in the cases of low pass filters, since higher frequencies stages vary, the signal conditioning circuit 503 eliminates the excessive bandwidths and the noise associated with it that can cause a distraction and either a false reading or an inaccurate allergy reading.

The remaining internal components were discussed in FIG. 4. In addition, other hardware components that aid to the operation of the IR module 400 are also contemplated.

The IR module 400 communicates to the server 103 to provide and receive data. In one example, the IR module 400 may communicate the results of the allergy test, results of particular allergen or wheal that resulted from the reaction to that allergen, or the panel to the server 103. In response, the server 103 may send a command for the camera 107 to be activated. Alternatively, the user may manually activate the camera 107 if an allergic reaction is detected.

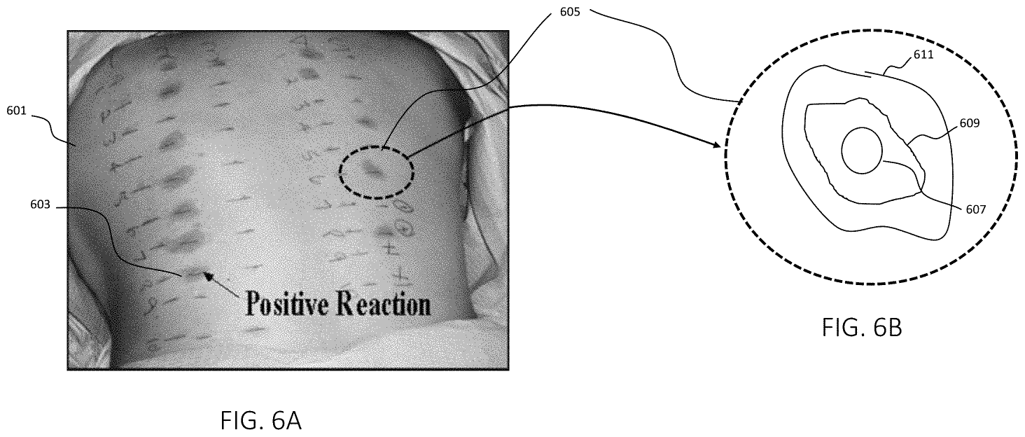

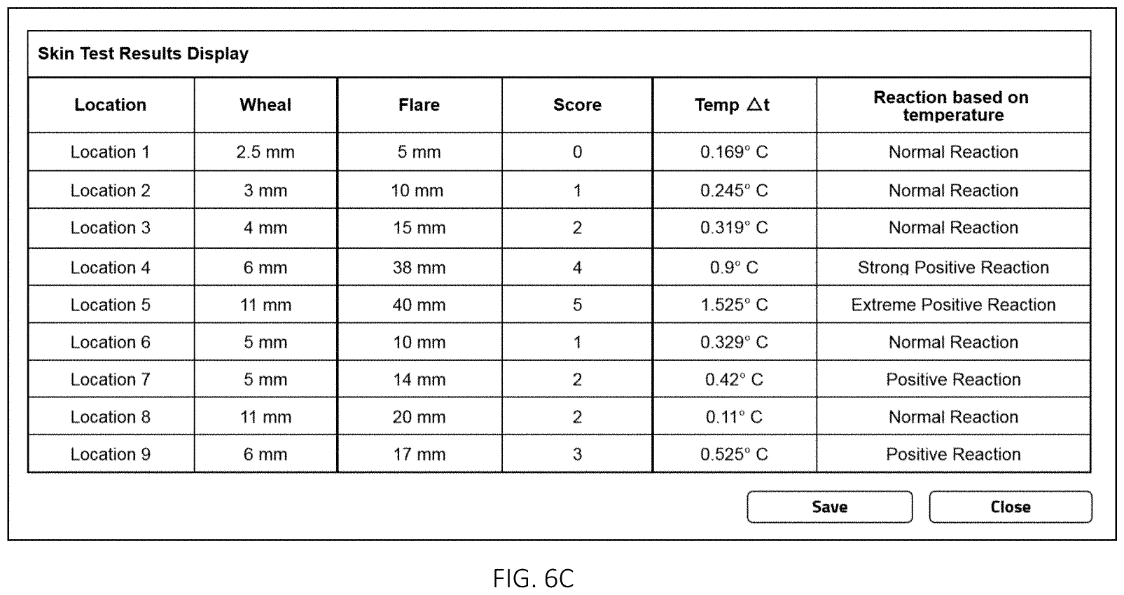

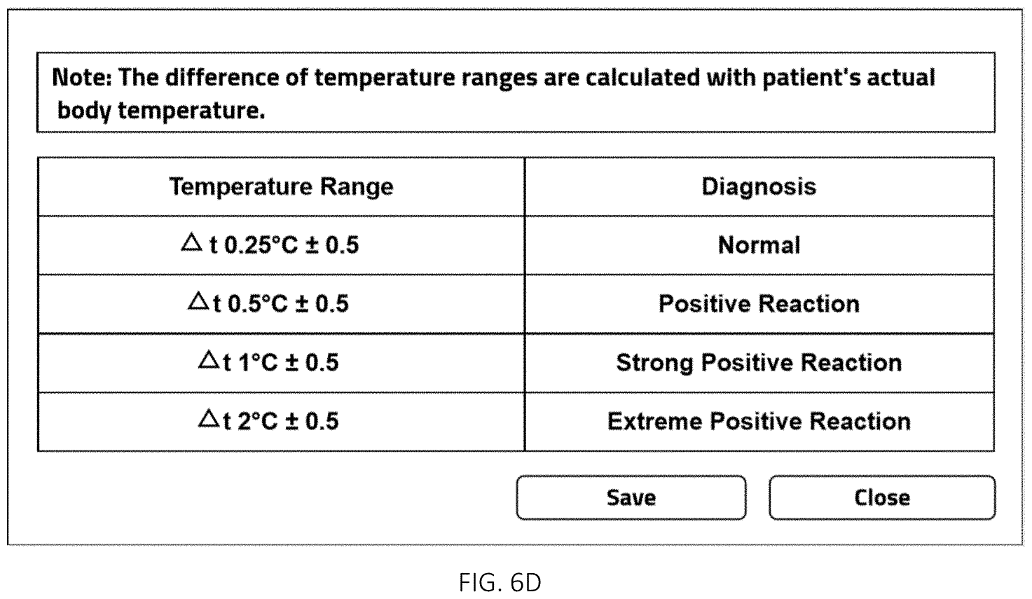

FIG. 6A illustrates the back of a patient's body with a plurality of wheals that occur as a reaction to the allergens injected according to the disclosed embodiments.

The grid is marked with numbers 1-10 on the left and the right side of the patient's spinal cord. There are both rows and columns, where rows are 1-10 and columns are 1-5.

The numbers are associated with an allergen that is injected in each space. For example, row 1, column 1 on the left side of the spinal cord may be an allergen that relates to a type of grass, such as crab grass, while row 2, column 1 on the left side of the spinal cord may relate to a type of weed.

The circles around each allergen are the wheals. As it can be seen in the figure, some numbers have a wheal around them and some don't. The wheal in 603 is a positive reaction, as are all the other wheals show. For numbers 9 and 10 on either side of the spinal cord, there are no wheals thereby indicating that the allergens injected in those areas does not have a positive reaction.

Also shown in this Figure is a wheal 605 that is in row 6, column 5, i.e. to the right side of the spinal cord. This is a positive reaction to an allergen injected in that space. For exemplary discussion, we will discuss this wheal in more detail in FIG. 6b. The readings for the wheal are shown in FIG. 6C.

FIG. 6B illustrates the single wheal and the density of a reaction that has occurred on the patient's body in response to an allergen injected according to the disclosed embodiments. The wheal 605 has different density of reaction. The shape and size depict the intensity of reaction. The inner circle 607 is where the rection's intensity is the highest and the reaction has dissipated or is less intense at circle 609 and even less at circle 611.

These radiations emanate from the patient's body form these circular regions that are read by the IR Module 400. The IR Module 400 is capable of reading the circles and take associated measurements of the wheal and the intensity. It does so by measuring radiation and temperatures of the wheal and its circles. The camera is also used to take an image of the circles and correlate them with the measurement taken by the IR Module 400. For a larger reaction, the wheal measurement is larger in shape while the wheal is smaller in a smaller reaction.

FIG. 6C illustrates an exemplary detail snapshot of a wheal that includes measurements, scores, and temperatures according to the disclosed embodiments. The wheal 605 as discussed in FIGS. 6A and 6B above could have a wheal measurement of 5 mm, flare of 10 mm, score of 1, and temperature delta of 0.329 C. The system would review all the readings and produce a result, which in this case is a normal reaction. However, as it can be seen in FIG. 6C, locations 4 and 5 have a stronger reaction.

FIG. 6D illustrates a temperature range and its associated reaction as calculated by the system according to the disclosed embodiments. As mentioned in FIG. 6C, the system produces a result based on the temperature ranges. An exemplary temperature range used by the system is depicted here in this figure. The temperature ranges may vary and adjusted as needed or the system may use a default. Each temperature range corresponds to the severity of the reaction.

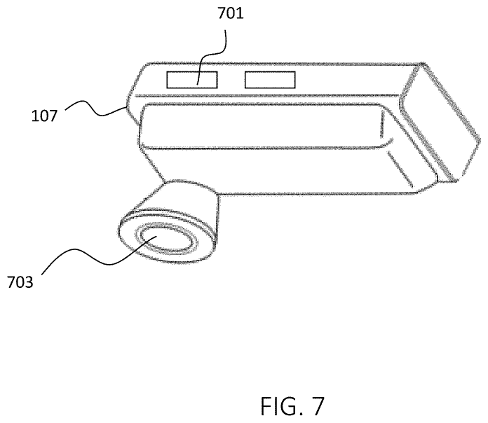

FIG. 7 illustrates a handheld camera used for capturing an image of a wheal according to the disclosed embodiments. The handheld camera 107 includes is a visible light camera that has a lens for capturing the image of wheal. The handheld camera 107 also includes a processor that allows the image to be transferred to the server 103.

In operation, the server 103 acquires the image from the handheld camera 107 and performs image processing. The server 103 communicates with the computing device 105 or a handheld electronic device 104 to display the image dimensions and temperature. It may also connect with an electronic health record (EHR) machine to send the data. The readings and results are depicted on the receiving device's display.

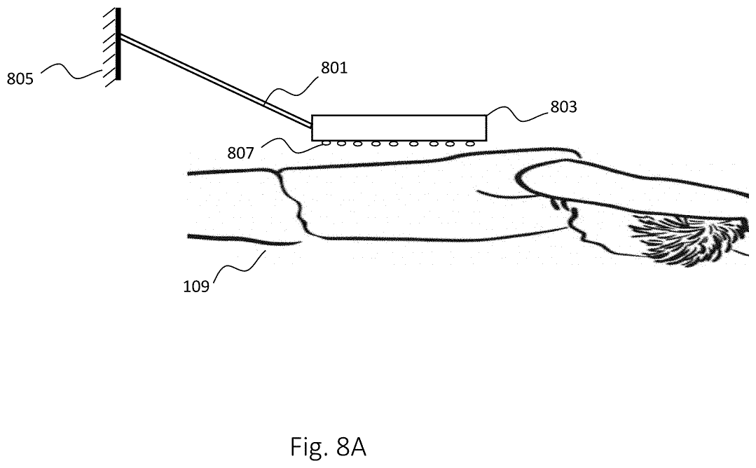

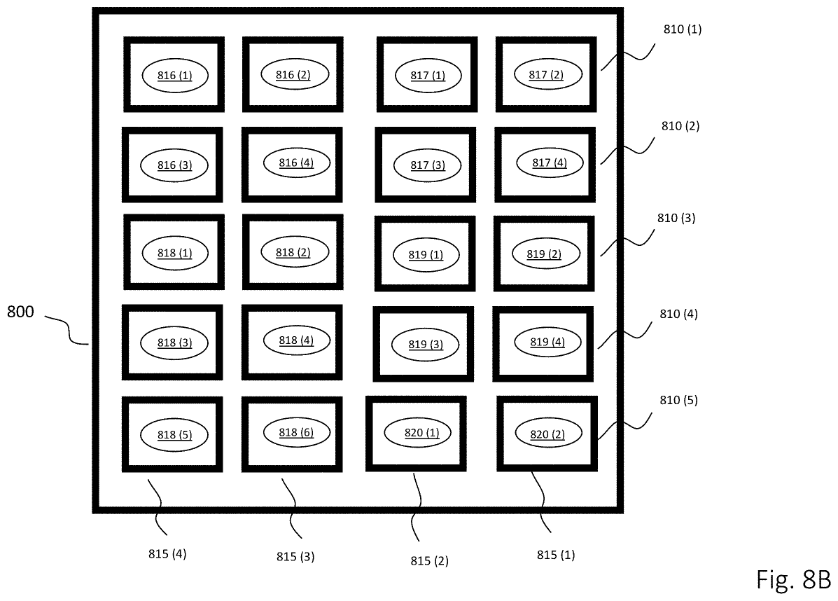

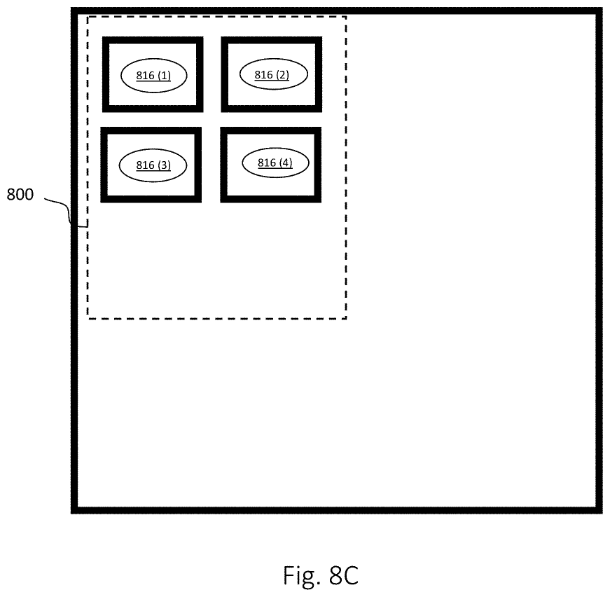

FIG. 8a illustrates an automated and secured camera used for capturing an image of a wheal according to the disclosed embodiments. The camera system 800 is used for capturing images of the wheal that may occur due to an allergic reaction to an allergen placed on the person's body. The camera system 800 includes a movable arm 801 and a camera body 803. The movable arm 801 is secured to a wall 805 or other support. The movable arm includes wires that provide an electronic connection and power to the camera. The wires and internal mechanism in the arm allow the arm to move, tilt, and be placed directly above the person's body 809. The movements of the movable arm 801 is directed by the server 103.

The camera body 803 includes a plurality of lens compartment in which a lens 807 is housed. The camera body may include a grid of lenses or just a few lenses, it depends on the need and various number of lens combinations are contemplated.

FIG. 8b illustrates a detailed cross-section of the camera of FIG. 8a and its lens according to the disclosed embodiments. Camera body 800 includes a plurality of lens compartments. Each compartment is configured to house and secure a lens that can be used for image capturing. Lens compartments 816 (1)-816(4), 817 (1)-817(4), 818 (1)-818(6), 819 (1)-816(4), 820 (1)-820(2), are arranged in rows 810(1)-810(5) and columns 815(1)-815(4). Although a panel of 20 Lens compartments and 20 lenses housed in them are depicted, it is noted that the number are shown for explanatory purposes only, and the actual implementation can have additional, or fewer rows and/or columns, and less or more lenses as needed and desired.

The lens compartments 816 (1)-816(4), 817 (1)-817(4), 818 (1)-818(6), 819 (1)-816(4), 820 (1)-820(2) are spaced a certain distance from each other thereby allowing each of the lenses to have their own field of vision and be able to capture the image of an individual wheal. In some implementations, only a few of the lenses may be used and in other implementations all of the lenses may be used to capture wheal images.

In one embodiment, the number of lenses used correspond to number of allergens placed on the body that need to be analyzed. Since each lens captures an individual image, the number of lenses used are determined by the number of allergens placed on the body.

The lenses may also be grouped such as lenses 816 (1), 816(2), 816 (3), and 816(4) are grouped together as a panel for 816. Likewise, 817 (1), 817(2), 817 (3), and 817(4) are grouped together as a panel for 817. Also 818 (1), 818(2), 818 (3), 818(4), 818 (5), and 818(6) are grouped together as a panel for 818, and 819 (1), 819(2), 819 (3), and 819(4) are grouped together as a panel for 819 and 820 (1) and 820(2) are grouped together as a panel for 820. In some cases, its common to group 4 or more lenses together and associate those lenses to a panel of 4 allergens to capture their images, however, a variety of combination is possible.

FIG. 8C illustrates a detailed cross-section of the camera of FIG. 8a and a selected panel of lenses according to the disclosed embodiments. In this embodiment, the lenses 816 (1), 816(2), 816 (3), and 816(4) are associated with a panel of allergens placed on a patient's body. For example, a 4-allergen panel may include 4 separate allergens that relate to a common theme, such as grass, foods, pollen, or another category. All the 4 allergens may differ from each other in some way but still be related to the theme—e.g. a food panel of nuts testing may include peanuts, cashews, almonds, and pine nuts—all relating to the nuts category but differ from each other.

In this example, lenses 816 (1), 816(2), 816 (3), and 816(4) would capture each separate wheal for a nut allergen placed on the body. Lenses 816 (1) may capture the wheal or reaction if any that resulted from the allergen associated with peanuts, 816 (2) may capture the wheal or reaction if any that resulted from the allergen associated with cashews, 816 (3) my capture the wheal or reaction if any that resulted from the allergen associated with almonds, and 816 (4) may capture the wheal or reaction if any that resulted from the allergen associated with pine nuts.

Each of the lenses would capture an image associated with the reaction or the wheal that results from the reaction. A sample reaction was shown in FIG. 6b. It may also be the case that no reaction has occurred for a specific allergen. For example, 816 (1), 816(2), and 816 (3) may have had a reaction while and 816(4) did not have a reaction at all. Also, it may be the case that 816 (1) had a severe reaction while the reaction captured by 816(2), 816 (3), and 816(4) were either minimal or none—it all depends on a case by case basis and differs from person to person. The camera technology provides image enhancements and clarity to ensure that the image captured is clear, readable, and includes all the data points needed for the server 103 to analyze and measure the reaction.

The camera and panels of the camera as discussed in FIGS. 8A-8C can be manually operated or automatically operated through the server. In one embodiment, the server allows the camera to auto-detect the allergen grid and then moves the camera arm such that the camera lenses are placed above the allergens injected areas of the body.

Quality assurance and verification of the image may be performed by the server 103 and in some cases the image may have to be recaptured. For example, if the image is not clear or if there is an overlap from an adjacent wheal, then the image may need to be recaptured.

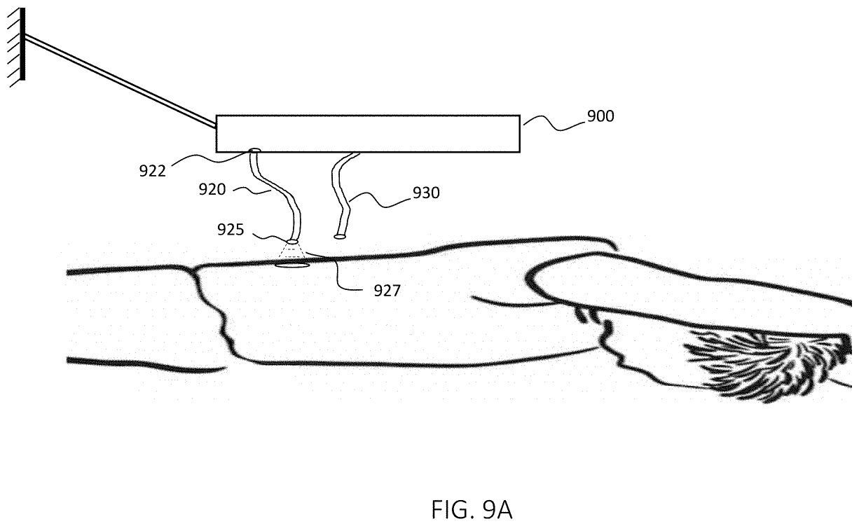

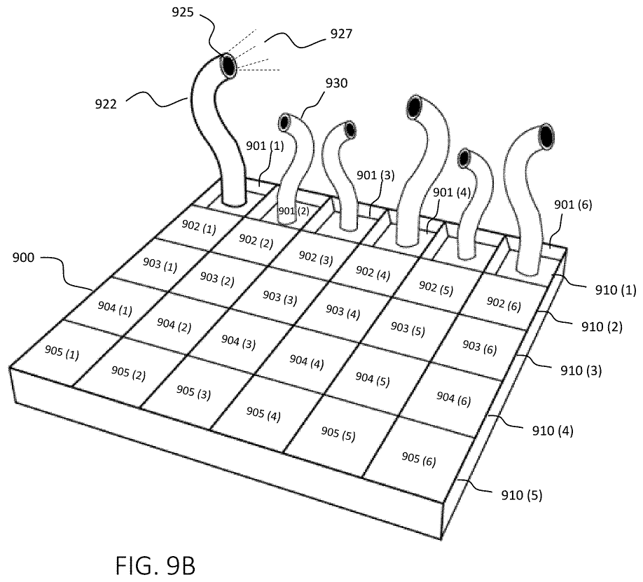

FIG. 9a illustrates an automated and secured camera having a plurality if movable prongs placed on top of the body to capture the image of a wheal according to the disclosed embodiments. The camera system 900 is described in detail in FIG. 9b. In this embodiment, the camera system 900 is depicted placed a certain distance from the back of a person's body to capture the images of wheals in their back. The wheals may be generated as a reaction to allergens placed on the body. The distance between the body and the camera 900 can be determined by the skilled nurse or can be automated through the server such that the server determines the distance needed for a clear capture of the wheal's image.

FIG. 9b illustrates a detailed view of an automated and secured camera having a plurality if movable prongs according to the disclosed embodiments. As mentioned, FIG. 9b depicts an automated and secured camera as shown and placed over a person's body in FIG. 9a.

Although similar to the usage and mechanism described in FIGS. 8A-8C. the camera system 900 of FIG. 9 differs in having individually adjustable prongs and camera lenses that protrude outwards for a closer and enhanced reading of the wheal.

Camera body 900 Each compartment is configured to house and a lens compartment and each lens compartment has a movable and flexible wired-like prong that protrudes and extends outward. The lens compartments 901 (1)-901(6), 902 (1)-902(6), 903 (1)-903(6), 904 (1)-904(6), and 901 (5)-905 (6), are arranged in rows 910(1)-910(5) and columns 901(1)-915(5). Although a panel of 30 lens compartments and 30 movable and flexible pronged lenses housed in them are depicted, it is noted that the number are shown for explanatory purposes only, and the actual implementation can have additional, or fewer rows and/or columns, and less or more lenses s needed and desired.

The flexible and movable prong 920 and 930 includes a base 922, the wire-like bendable body, a tip 925 that includes a camera lens that can focus on a field of vision 927. The flexible and movable prong 920 is used for enhanced focal illumination to capture an image of a specific wheal. The bendable prong can be retracted and protruded forward as desired—i.e. its length may also be adjusted based on the body curvature. The flexible and movable prong 920 and 930 allows both focus and zoom into the wheal as need. The image is enhanced such that it is not pixelated or unclear due to the zoom. Both flexible and movable prongs 920 and 930 can be adjusted individually and separate from each other such that 920 can point in a different direction than 930.

The tip can orient in relation to the body curvature. The lens is housed within the tip and includes a means for retaining the lens such that is captured within the housing of the tip. Antifogging mechanisms may also be used to ensure the camera lens does not gets fogged or get unclear due to the surrounding conditions or due to the heat generated by the body or the reaction.

The system includes an application executed on the server 103 that manages the movement of the prongs individually or as a panel. The user may also adjust it manually or through an interface that is provided on the computer, laptop, or connected mobile device.

Since the human body has curvature and areas that are not uniform, in one embodiment, the grid of allergens placed on back of the body needs to be manually placed thereby having the operator or the skilled nurse to place the allergens various distances apart from each other such that enough and adequate space is provided between the allergens to distinguish one reaction from another reaction. If the allergens are place too close together, then there may be reaction or wheal overlap that makes the results inaccurate or inconclusive.

As such the skilled nurse will place the allergens following the contours and curves of the body while adhering to proper spacing requirements, e.g., more than 1-inch or more than 2-inches between any 2-allergens. To closely follow the allergens grid, in which each allergen is not an equidistant away from each other, or if it is equidistant, then may be in on a curve of the body or in a ridge thereby making it farther in a 2D planar measurement, the camera lens needs to be adjusted. In case of a rigid camera grid, the cameras may be fixated to the camera body such that they may not allow any motion in between the lenses thereby allowing each individual lens to follow the body contours and focus on the wheal. As such, the prong camera depicted has movable and flexible prongs. One end of the prong is securely fixed inside the body of the camera while the remaining length of the prong and open end of the prong is capable of bending, flexing, and twisting. The bending and twisting of the prong allow the lens that is housed inside the prong to be adjusted such that it is aimed only on a specific wheal whose image needs to be captured.

Although the lens compartments 901 (1)-901(6), 902 (1)-902(6), 903 (1)-903(6), 904 (1)-904(6), and 901 (5)-905 (6) are spaced a certain distance from each other, the prong that extends outwards allows the distance between the lenses to be adjusted either manually or automatically through the guidance by the server 103. Once adjusted, each pronged flexible lens has their own field of vision and be able to capture the image of an individual wheal. In some implementations, only a few pronged flexible lens may be used and in other implementations all of the lenses may be used to capture wheal images.

In one embodiment, the number of pronged flexible lens used correspond to number of allergens placed on the body that need to be analyzed. Since each lens captures an individual image, the number of lenses used are determined by the number of allergens placed on the body.

Similar to FIG. 8a-c, the pronged flexible lens may also be grouped together and associated with a particular allergen panel placed on the body. E.g. a panel of grass may include 3 or 4 different varieties of grass and each pronged flexible lens may be associated with one of the 4 varieties of allergens associated with each type of grass placed on the body to capture their image. The pronged lens panels of described in the figures above can be used such that the IR Module and the system is capable of capturing a plurality of wheals, that occur due to allergic reaction, and their reading at an instant and in real time. The processor of the system would be capable of associated each prong to a specific wheal and compute all the reactions instantly along with correlating each reaction with the camera to provide a full panel report instantly and in real time as the reactionary changes occur and the wheals continue to change their shapes during the testing process.

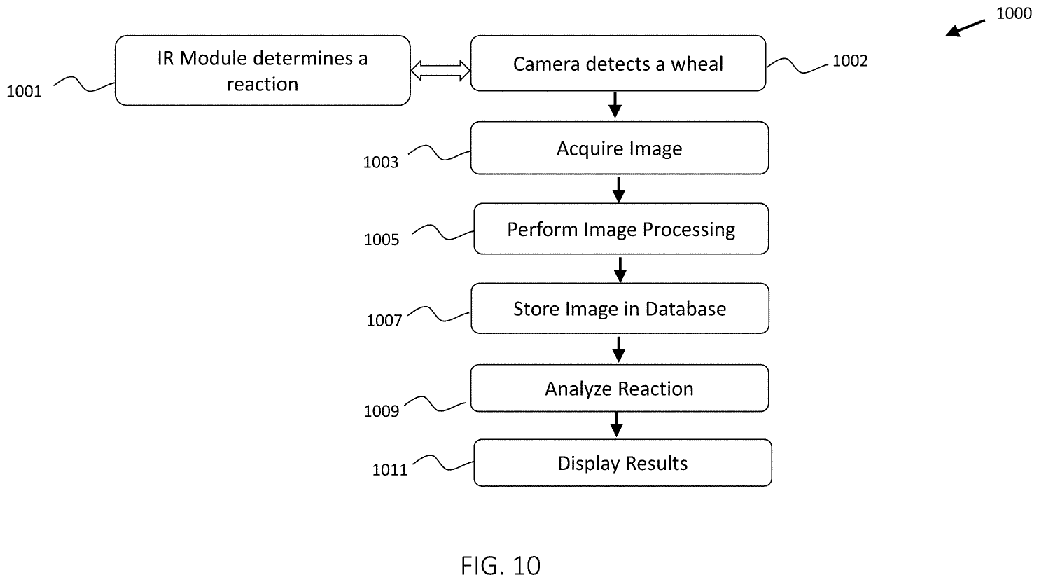

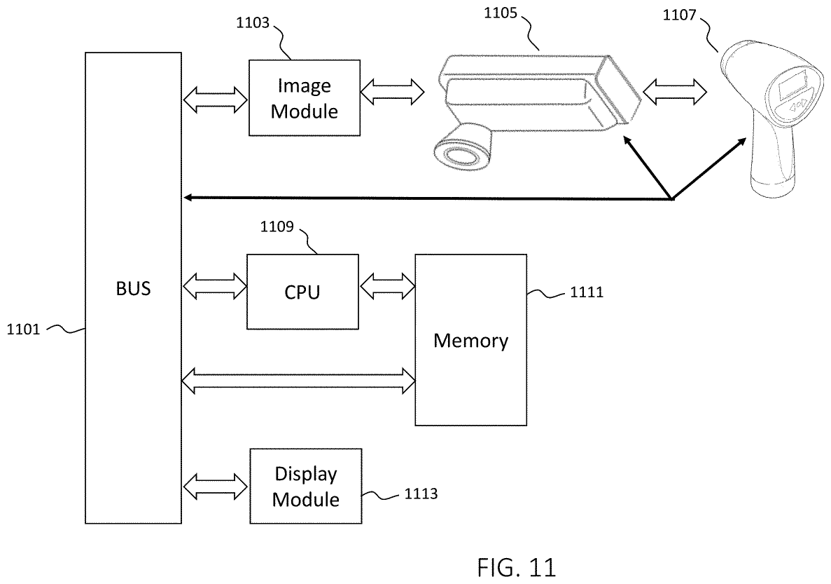

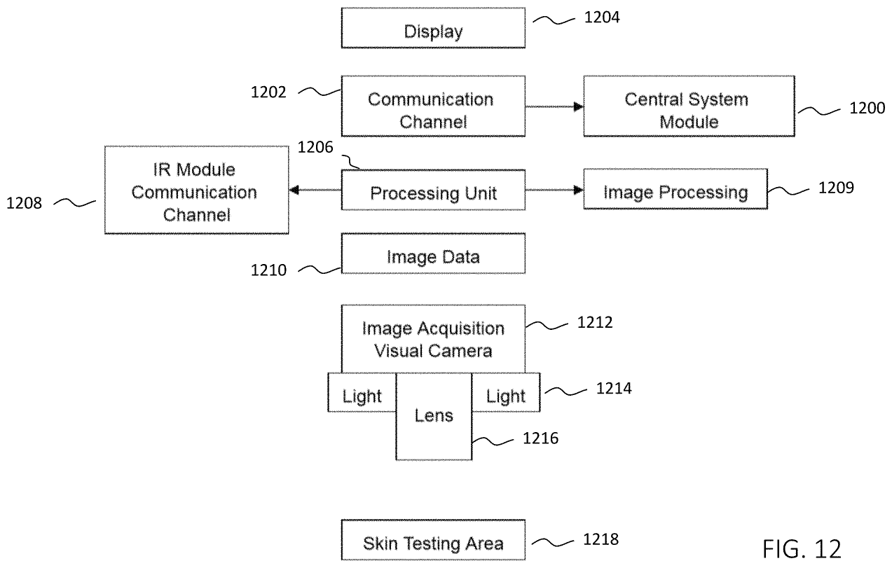

FIG. 10 illustrates a block diagram depicting one exemplary process of reading and analyzing a wheal formed due to an allergic reaction according to the disclosed embodiments. The process 1000 uses the system depicted in FIG. 11. Process 1000 can also use the systems depicted in FIGS. 1 and 2.

At step 1001 the process 100 starts with the IR module 109 detecting a reaction to one or more of the allergens placed on the body. The reaction occurs in the form of a redness of the skin as well as itchiness. The person on whom the allergens are injected may also have visible hives and other forms of redness. In some cases, the reaction may also be only irritant reaction and not an allergic reaction. Whatever the form of reaction may be, if there is a redness, rash, hives, or heated area, the IR module 109 will detect that a reaction has occurred. The reaction is then measured and quantified.

Once the IR module 109 confirms the reaction, i.e. that there was a redness, hive, or a rash then it reports the reaction to the server 103. The IR module also reports the severity and intensity of the reaction that is based on the quantitative results to the server. As mentioned earlier, the result of the reaction is a wheal being formed. The server 103 in response to a confirmed reaction activates the camera 107. The camera may be one of the cameras depicted in FIGS. 1, 7, 8A-8C, or 9A-9B or another camera with similar features and capabilities.

In another alternative embodiment, the process may also start with the camera 107 at step 1002 instead of starting at step 1001. In this instance, the camera 107 would be the first device to detect a reaction and capture its image. For example, the camera 107 may take routine images of the person's body on different intervals after allergens have been injected into the person's body. The camera's image processor would then detect if a wheal image was captured and if so confirm that a reaction has occurred based on the dimensions of the wheal. Once a reaction has occurred, if it has not already been processed by the IR module 109, then the server 103 may activate the IR module 109 or alert the operation to measure the reaction with the IR module to determine its severity and quantity the reaction.

At step 1003 an image is captured and acquired. Whether the process starts at step 1001 or at step 1002, in either case the image of the wheal is captured at step 1003. The image capturing occurs with the lens of the camera taking a photograph or scanning the wheal to capture the entire periphery of the wheal. The camera may have to focus or be moved closer to the wheal in certain instances to capture the reaction.

At step 1005 the image is processed by server 103. Alternatively, the image may be processed by another computing device or workstation that is associated with the system 100, as shown in FIG. 1. The image processing includes determining the clarity, completeness, blurriness, color, opacity, and capture of the contours of the wheal. Many methods such as anisotropic diffusion, Markov models and similar digital image processing methods may be applied. The processing includes determining if image dimensions and temperature have been captured accurately. The processing includes use of computer algorithms for the purposes of obtaining an enhanced image and extracting useful information from the image, e.g. in the case of a reaction, the useful information would be dimension of the wheal and the temperature. The image pixels are also analyzed to determine if the image is pixelated, thereby resulting in an unclear image.

The communication between the image acquisition system and the server or processing unit is done by the communication channel. In one embodiment it may be through a secured wireless pairing of the two systems. The processing unit may also include a user interface. This interface can be manually operated by a skilled technician to tweak the processing during the image capture or to retake the image. The interface also allows the user to utilize the downloaded software from the EMR or server and use the features of the software in image capture and processing.

If the image for any of the reasons discussed above is not approved after it has been processed, then the system 100 would alert the user to capture another image and replace it with the prior taken image. The server 103 may also automatically activate the camera to take another image and likely change the position or focus lens parameter to capture a better image.

Once the image capture and the processing are approved by the server 103, the image is stored in the database 207. The storage may also be on an optical disk, tape, videotape, magnetic tape, a connected electronic device or in the memory of an electronic medical record (EMR) module.

At step 1009 the reaction is analyzed. A plurality of analyses may be performed to dissect the reaction into various components. One such analysis includes comparing the reaction to a previous reaction to determine whether the severity has increased or decreased. Another analysis would be to determine the type of reaction and quantify the severity. Yet another type of analysis would be to determine if another allergen should also be tested. The analyses are further discussed throughout this application.

The results of the analyses are then displayed at step 1011 on a display. The display may be part of a computing device, an electronic module, an EMR system, or a system computer that is authorized and connected to the system 100.

FIG. 11 illustrates an architectural block diagram depicting the system used by the IR module and camera according to the disclosed embodiments. As show in the figure, the bus 1101 connects the components 1103-1113 in the system. The IR module 1107 and camera 1105 interact with each other as well as with the bus to obtain readings of a wheal and acquire an image. As discussed in FIG. 10, the bi-directional communication between the camera and the IR module is to ensure that both steps of reading are performed. The two types of readings, i.e. the image capture of the wheal by the camera and the measuring of the reaction, together provide a complete data capture of an allergic reaction. The two components, IR module and camera, working with each other also provide verification of the allergy and eliminate any errors or false positives. For example, based on the image captures of the wheal, the server or processing unit may provide a certain result that is indicative of the type of allergy and its severity. As a secondary check, the system may also compare the data obtained by the IR module to determine if the result calculated based on the camera image capture is in accordance with the IR module data. If the result based on camera data is outside a predetermined range of possibilities based on the IR module data, then the system determines that an error has occurred in one of the readings and both readings are performed again. As such, both camera and IR module also act as a secondary verification mechanism.

The image module 1103 is communicatively connected with the camera 1105 and performs all the image processing, if an error is found, it alerts the camera 1105 or the operator to take a second image.

The CPU 1109, memory 1111, and display 1113 units are used by the system to process data, store data, and display results.

FIG. 12 illustrates a block diagram depicting the central system module and its communications and connections with a plurality of physical and computational objects that are part of the system according to the disclosed embodiments.

In various parts of the application, reference has been made to an algorithm or software powering being downloaded onto the server 103 or residing on the computing device 105 and interacting with the server 103. It may also reside on a separate device or computer that is connected to the server 103 through the network 111. It may also reside on in the database 207. The software, which manifest in the decision logic and complex architecture, may also be downloaded onto an electronic medical records (EMR) machine or an alternative medical health records machine.

In one embodiment, the server 103 houses the decision logic and complex computational objects and components that provide for complex navigation through decision trees to analyze allergy reading, analyze reactions, determine course of action, and provide allergy management solutions, such as medication intake, medication management vaccine management, and vaccine scheduling.

For sake of simplicity, the decision logic and complex computational objects and components are herein referred to as central system module (or also IMS) 1200. The central system module provides communications and interactions between the physical components as well as computational objects residing on the physical components using the decision logic. The physical components referred to herein include, but are not limited to camera 107, arm 106, IR module 109, server 103, computing device 105, mobile device 104, and other physical and computational elements depicted in the Figures through this application.

Using the decision logic, the physical components as well as computational objects residing on the physical components are able to perform a variety of functions. These include, but are not limited to, the operation of the camera, the IR module, the transferring of data between them, performing readings of a wheal, analyzing the results, operationally moving the physical objects such as the arm for the camera, adjusting the focus of the IR module and other operations described through the application.

Some additional functionality of the central system module 1200 includes a) treatment or containment of allergy, b) determining the root cause and triggering factors of the allergy, c) analyzing various factors of any allergic reaction caused due to the deposition or injection of allergens on the body, d) using a processing device to analyze and process all the data obtained during the course of allergy skin test including managing temperature readings and wheal and flare diameter data and transmitting the data to from one module to another module as needed and processing the data to display the measurement in immunotherapy skin associated with each allergen on a user interface, e) operating all features and functionality of the IR Module, including the activation of the IR module, the heat modulation, and the reading of the wheal, e) operating all features and functionality of the various types of cameras along with its arms as depicted in FIGS. 7, 8A-8B, and 9A-9B, and g) performing image processing on the image acquired through the cameras depicted in FIGS. 7, 8A-8B, and 9A-9B.

As shown in FIG. 12, the central system module 1200 is communicatively coupled to components 1202-1218 of the system 100. The communication channel 1202 serves as a communication interface between the central system module 1200 and components 1204-1218.

The processing unit 1206 is used for processing all the data and performing analyses of allergic rection as well as any other allergy related analysis that is to be performed. For example, if a newer allergy test is to be compared with an older allergy test, the processing unit 1206 would perform such analysis and comparison. Likewise, if external data is needed to determine the root cause of allergy or external data for some outbreak or pollen count, then the processing unit would obtain such data through network 111 and then process it in comparison to the patient's allergy skin test or allergy data.

The IR module 1208 and camera 1212 with its components lights 1214 and lens 1216 is operated by instructions provided form the central system module 1200. Data captured by the IR module 1208 and camera 1212 for the patient located in the skin testing area 1218 is also acquired by the central system module 1200 and send to appropriate modules, such as processing unit 1206 or image processing module 1209 for processing and analyses. The central system module 1200 would provide instructions for a second or subsequent readings by the IR module or camera if any errors or inaccurate readings are detected by the processing unit 1206 or the image processing module 1209.

All the results are displayed on a display 1204. The central system module 1200 also provides a user interface to both display the results of the analyses and various allergy parameters and details to the user as well as to receive user input during all allergy testing, analyses, and management operations.

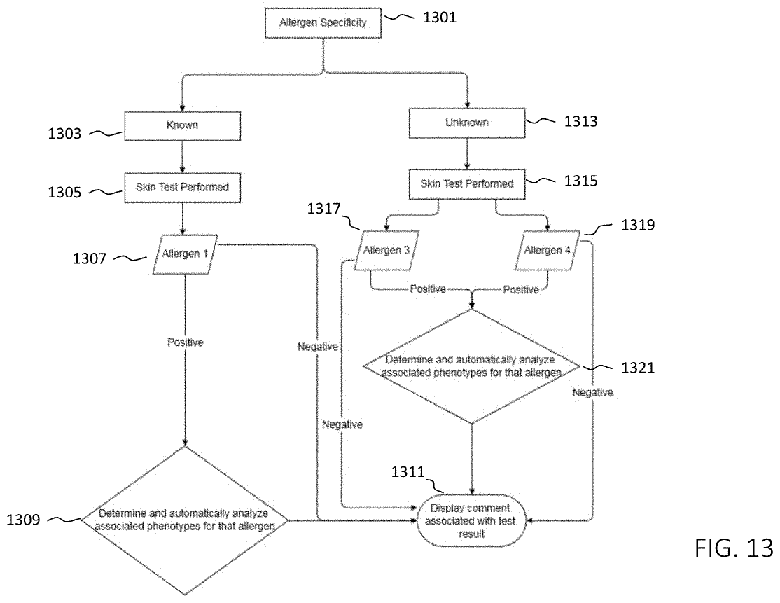

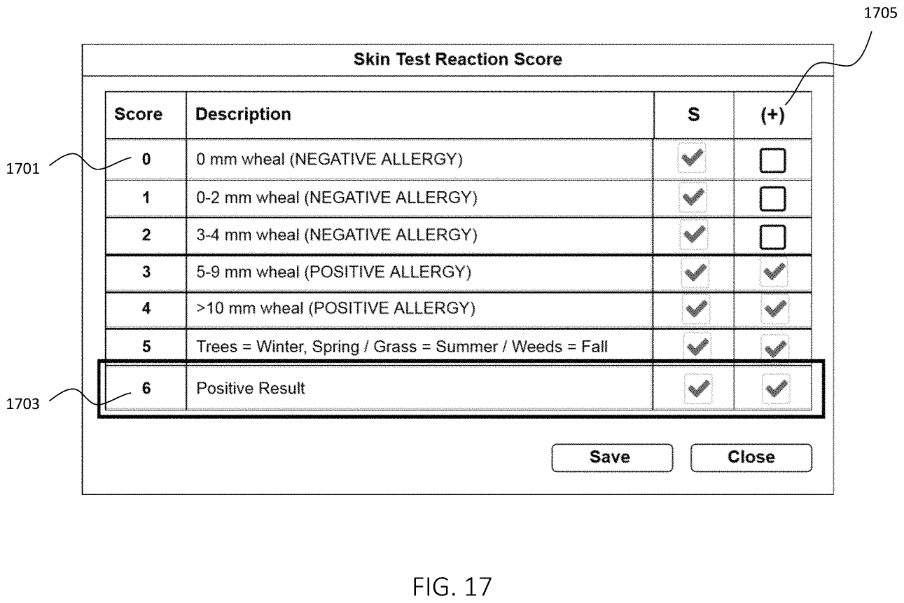

Some of the analyses performed by the central system module 1200 are discussed in FIGS. 13-15 below and some of the user interface features, operations, and displays are discussed in FIGS. 17, 20-25 below.

FIG. 13 illustrates a block diagram depicting one exemplary process of the central system module determining a phenotype for an allergen and displaying test results according to the disclosed embodiments.

At step 1301 the central system module 1200 determines the allergen specificity. This process involved determining whether the type of allergen tested is one of the known allergens that is known to cause a reaction or allergy to the patient. It could also mean that a certain specimen to which the patient is allergic to it brought forth and the type and details of the allergy specimen as well as its specificity is not known and needs to be determined. It could also mean that the allergen composition of molecules that provide the desired specificity that would cause a reaction to the patient or the specificity when compared with an allergen extract-based assays is either not known to the specific molecular detail level.

At step 1303, if the allergen specificity is known, or becomes known after some research or comparison of data stored in the server's database, then at step 1303 the central system module 1200 concludes that the specificity of the allergen is one of the known types.

At step 1305, a skin test with the allergen is performed with the allergen and the data from the skin test is obtained by the central system module 1200.

At step 1307, the central system module 1200 determines whether the allergen used to perform the skin test resulted in a positive or negative reaction. If the result was positive, i.e. that a reaction occurred thereby forming a wheal on in the area where the allergen was injected into the patient, then at Step 1309, the central system module 1200 determines and automatically analyses the associated phenotypes for that allergen.

The analysis to determine the phenotypes relates to determining the set of observable characteristics of the patient that result from the interaction of its genotype with the environment. Determining and identifying the right phenotype is important for performing an accurate investigation of underlying patho biologic mechanisms that can be used for providing specific and accurate treatment that would help in preventing or limiting the allergic reaction or properly managing the allergy that is being caused by the allergen.

At Step 1311, if there is a negative reaction, the central system module 1200 displays the test results along with any comments and details relating to the allergen and phenotype analyzed. The results are also displayed if the process navigates through step 1309 instead of step 1307.

After step 1301, if the allergen specificity cannot be determined at step 1303, then at step 1313, the central system module 1200 concludes that the specificity of the allergen is unknown.

This process of determining whether the specificity is known or unknown, if the previously conducted test on the patient resulted in a positive reaction, or a certain specimen to which the patient is allergic to it brought forth and the type and details of the allergy specimen as well as its specificity is not known and needs to be determined, all such determinations involve the central system module 1200 to analyze the allergen and compare it with a library of allergens previously tested to determine a match or to compare previous tests and their results. The library stored in the database may include numerous allergens that were either previously tested or known to cause a reaction or allergy to the patient.

The allergen specificity may also be unknown because a certain specimen to which the patient is allergic to it brought forth and the type and details of the allergy specimen as well as its specificity is not known and needs to be determined. It could also mean that the allergen composition of molecules that provide the desired specificity that would cause a reaction to the patient or the specificity when compared with an allergen extract-based assays is either not know or not know to the specific molecular detail level.

At step 1315, skin test with the unknown allergen specificity is performed and the data from the skin test is obtained by the central system module 1200.

At steps 1317 and 1319, Allergens 3 and 4 are tested on the patient. At steps 1317 and 1319, the central system module 1200 determines whether the allergens 3 and 4 that were used to perform the skin test resulted in a positive or negative reaction. If the result was positive, i.e. that a reaction occurred thereby forming a wheal on in the area where the allergen was injected into the patient, then at Step 1321, the central system module 1200 determines and automatically analyses the associated phenotypes for that allergen.

At Step 1311, if there is a negative reaction to either allergen 3 or 4, the central system module 1200 displays the test results along with any comments and details relating to the allergen and phenotype analyzed. The results are also displayed if the process navigates through step 1321.

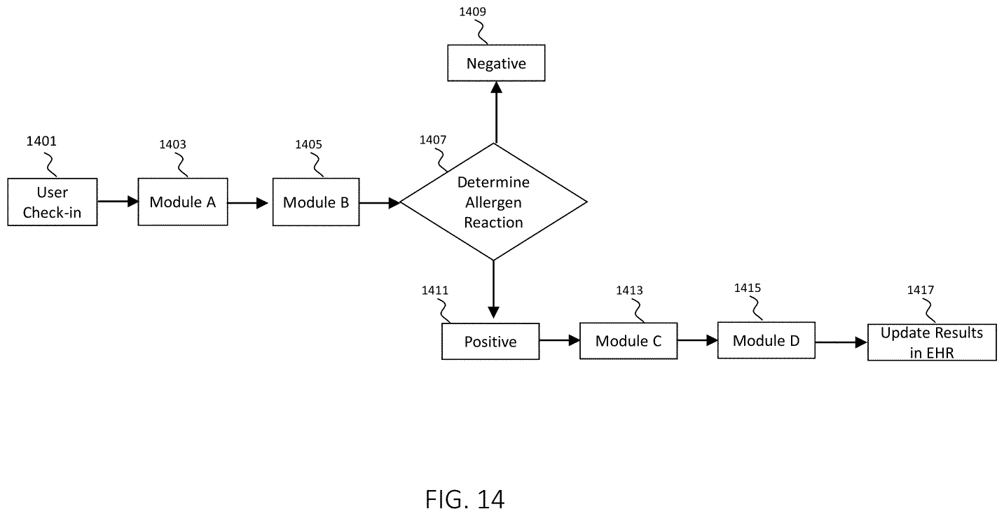

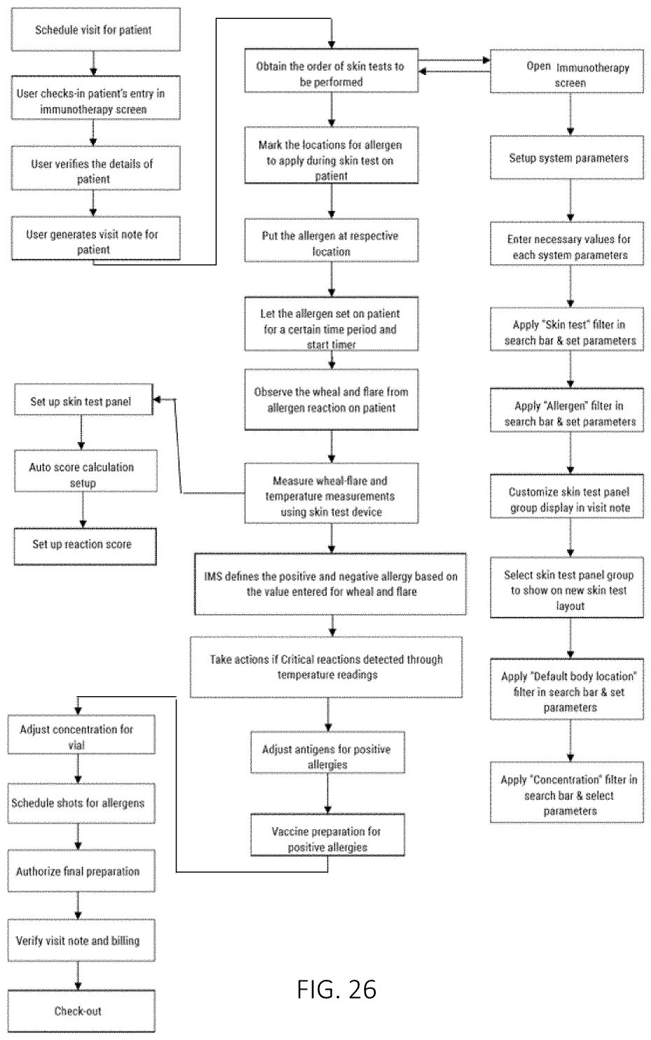

FIG. 14 illustrates a block diagram depicting one start-to-finish cycle of performing a skin test and updating an electronic health record according to the disclosed embodiments. Analogues and similar to FIG. 14 is another process as depicted in FIG. 26.

At step 1401, a patient or a person that needs to be tested for allergies checks into a facility (e.g., clinic, medical facility, or an institution that is skilled and authorized to perform allergy testing). Either a receptionist, front desk person, or a skilled operator enters data into the central system module 1200. The data includes the patient information and that they checked into the facility. The receptionist, front desk person, or a skilled operator may also simply pull up their previous record if they have been a patient of the facility.

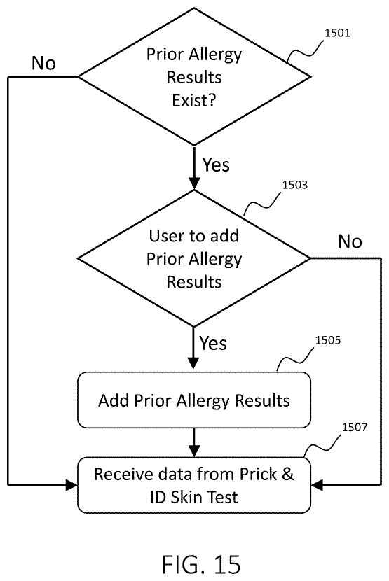

At step 1403, central system module 1200 activates Module A. FIG. 15 illustrates a detailed submodule A (also referred to as Module A) that is used as part of the exemplary start-to-finish cycle described in FIG. 14 according to the disclosed embodiments.

Once the central system module 1200 activates Module A, at Step 1501, the central system module 1200 determines if prior allergy results exist. It does so by checking the database associated with the central system module 1200, such as database 207. The prior allergy results may also be saved with the server or with an electronic health record system or with an electronic or computing device connected to the system 100. The prior allergy results may also be saved directly in a memory housed in the central system module 1200.

At step 1503, central system module 1200 finds prior allergy results for the same patient that is being tested and displays them on a user interface. The operator of the system, i.e. the skilled technician or medical professional, or the patient is provided an option through the user interface to choose to add the prior allergy results to the current tests being performed or to load them into the central system module 1200 such that they can be used for analysis, such as to compare new results with older results or other types of analysis to determine progression of allergy or management of allergy. Likewise, the skilled operator (or the patient or the medical physician) may choose to add symptom category for the patient's disease and known (previous positive results) allergen specificity.

At step 1505, upon the skilled operator choosing to add the prior allergy results, central system module 1200 loads the prior allergy results into the current test being performed or loads it onto the user interface such that they can be used for analysis as needed.

At step 1507, central system module 1200 receives data from the current prick and ID skin test.

At steps 1501 and 1503, if the skilled operator, or others as mentioned above, choose to not add the prior allergy results or if they do not exist, then also the central system module 1200 proceeds to step 1507 and receives data from the current prick and ID skin test.

Further to the above, the central system module 1200, if the operator or others choose not to include a prior skin allergy test, and such a test exists, then such a selection results in the central system module 1200 automatically generating a new test or screening and deleting or cancelling all prior allergy-related tests form the current testing data.

However, if the patient is tested positive for specific allergen previously, the associated phenotypes for that allergen will be automatically analyzed central system module 1200.

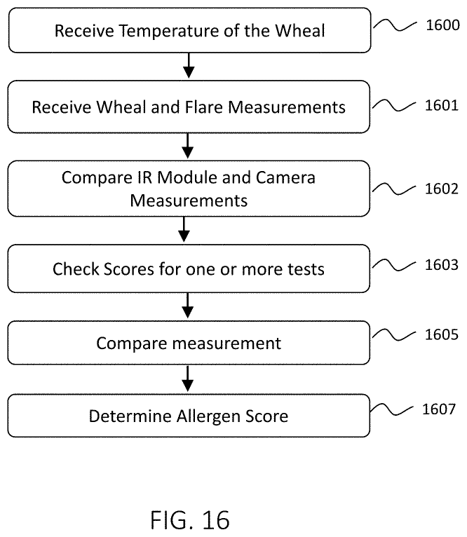

Referring back to FIG. 14, after steps of Module A have been performed, the central system module 1200 activates Module B. FIG. 16 illustrates a detailed submodule B (also referred to as Module B) that is used as part of the exemplary start-to-finish cycle described in FIG. 14 according to the disclosed embodiments. Module B also represents both IR temperature measurements and image dimensions captured by the camera.

At Step 1600, the temperature and the reading of the wheal are received. In some cases, prior to testing, the patient's body temperature may be taken.

At step 1601, a skin test is performed on the patient. The central system module 1200 receives the data associated with the test performed. In particular, the central system module 1200 receives measurements of the Wheal and Flare. The measurements are obtained using either IR module or camera or both the IR module or camera as described in FIGS. 11 and 12. At Step 1602, the camera measurement and the IR module measurements are compared with each other to determine if they are within the acceptable range and if the readings taken by either of the devices have an error—it's a process of validation and verifying and correlating results from one device to another as a secondary check.

The server, the IMS, the electronic module connected to the server, or an electronic health record machine may have stored one or more skin tests. Each skin test stored would have a score associated with it and the score would also be stored accordingly.

In one embodiment, the central system module 1200 determines a score between 0 and 6 for each allergen. The scores from prior allergy testing for each allergen are also scored similarly and stored. The score is associated a temperature reading for that particular wheal. In another embodiment, an interface that is shown to the user depicts a skin test panel window and a score is set and predetermined based on the value of temperature readings and wheal and flare dimensions which are provided by the user. For example, for a definite range of wheal measure corresponding to a definite range of flare measure, score is defined.

FIG. 17 depicts on exemplary scoring of allergens as displayed on a user interface that is communicatively coupled to the central system module according to the disclosed embodiments. As shown in FIG. 17, the allergens are scored between 0 (1701) and 6 (1703). The higher scores are indicative of a positive result, i.e., that an allergic reaction occurs or has occurred for the patient and is noted in the user interface at 1705.

Referring back to Step 1603 of FIG. 16, the scores are checked by the central system module 1200 and compared at Step 1605. In checking the scores, if a score or a temperature readings generated by the IR Module identifies a critical wheal which is indicative of a serious reaction that will require immediate action, an alert is produced for the patient to be taken to emergency at a hospital or for the technical or medical physician to take immediate measures to contain and control the allergic reaction.

The central system module 1200 also includes a prediction module that predicts if a reaction will reach the stage of an emergency or critical stage by monitoring the progression of the reaction. For example, if a wheal is forming at a much faster pace or if the temperature is rising at a much faster pace than an industry standard for a reaction to an allergen, then the, central system module 1200 performs calculations to determine of the reaction would reach the emergency or critical stage. If the calculations determine that such stage is likely to occur, then the central system module 1200 provides the alert prior to the reaction getting to its extreme stage as a preventative measure to take action. Further, the central system module 1200 may also automatically shut down any tests being performed.

At step 1605, central system module 1200 compares the scores of the current allergen or skin test to other tests performed. At step 1607, central system module 1200 determines a score for each allergen tested based on comparative measurements with predetermined threshold.

Referring back to FIG. 14, at Step 1407, the central system module 1200 determines whether a reaction occurs or has occurred due to the allergen used. The determination of positive/negative is based on the score of the allergen, which in turn is associated with the measured value and temperature of the allergen. The central system module 1200 then displays the positive negative results on a display such as the user interface. For example, if a negative result is determined at Step 1409, the central system module 1200 displays that no allergic reaction has occurred in connection with the allergen tested and displays it on the user interface.

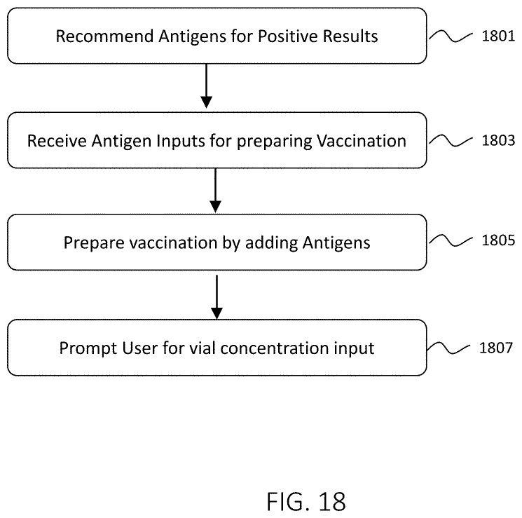

At step 1411, if a positive reaction is determined, then central system module 1200 activates Module C. FIG. 18 illustrates a detailed submodule C (also referred to as Module C) that is used as part of the exemplary start-to-finish cycle described in FIG. 14 according to the disclosed embodiments.

At step 1801, the central system module 1200 recommends an Antigen. The recommendations are based on the positive reactions—i.e. if a reaction has occurred.

In one embodiment, the user interface, based on the instructions of the central system module 1200 depicts a vaccine preparation screen. The central system module 1200 automatically recommends antigens and displays the recommended antigens on the user interface. The recommendation is based on which skin test was positive. Further, antigens which can be mixed together for vaccine preparation will also be recommended.

At step 1803, the central system module 1200 receives the antigen inputs for preparing the vaccination and then prepares the vaccines at Step 1805.

Prior to preparing the vaccines, the central system module 1200 performs an analysis to determine paring or antigens, whether or not to mix certain antigens, and storage of antigens in vials.

For example, if the antigen cannot be mixed with any other antigen, it will be shown separately and a recommendation will be made to not mix such two or more antigens together. For such antigens that cannot be mixed, they are prepared separately and kept separate in separate vials.

The central system module 1200 also analyses which pair or group of antigens can be mixed together along with details such as type and quantity of diluent for preparing the vaccine.

Additionally, for plural antigens and considering all the cases of which antigens can be mixed or not, the central system module 1200 will recommend the most preferred possible combination of antigens such that the patient can be given a minimum number of vial shots.

At step 1803, the central system module 1200 prompt the user for vial concentration inputs. In one embodiment, the central system module 1200 will cause the user interface to display a vial concentration mix screen. The central system module 1200 may provide suggested concentration and volume of individual antigens in the vial or ask the user for input. It may also allow the user to select the suggested concentration.

Using the user interface, in accordance with steps 1805 and 1807, the user is able to change the combination of antigens and adjust the concentration or volume of all the antigens or diluent. For example, if the patient is tested positive for 5 allergens and correspondingly, their antigens are A, B, C, D and E. If the antigens A, B, C, D can be mixed together in a vial with diluent and provided to the patient, it will be shown in the user interface for selection. However, if the antigen E cannot be mixed with any other antigen it will show separately.

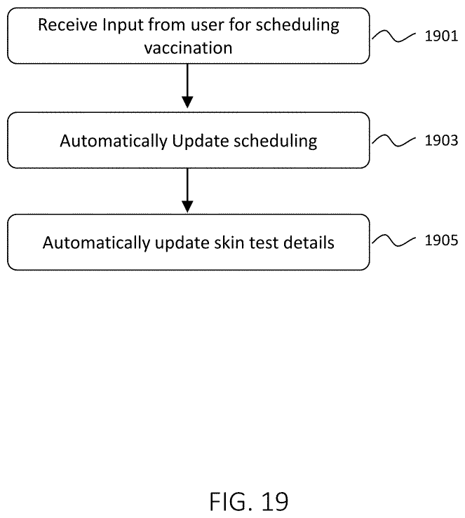

Referring back to FIG. 14, at Step 1415, the central system module 1200 activates Module D after completion of Module C. FIG. 19 illustrates a detailed submodule D (also referred to as Module D) that is used as part of the exemplary start-to-finish cycle described in FIG. 14 according to the disclosed embodiments.

At step 1901, the central system module 1200 receives input from the user for scheduling the vaccination. The scheduling options may allow the user to access the facility (clinic, hospital, allergy center etc.) calendar along with available times in real-time and allow the user to self-book the appointment for the vaccination. In the event the user cannot determine an open time slot, the central system module 1200 may provide next available date or open schedule.

At step 1903, the central system module 1200 automatically updates the vaccine scheduling upon the user selecting a date and time for coming into the facility to take the shot. The update is recorded and a corresponding technician or nurse is sent an update such that the appointment makes a booking on their calendar.

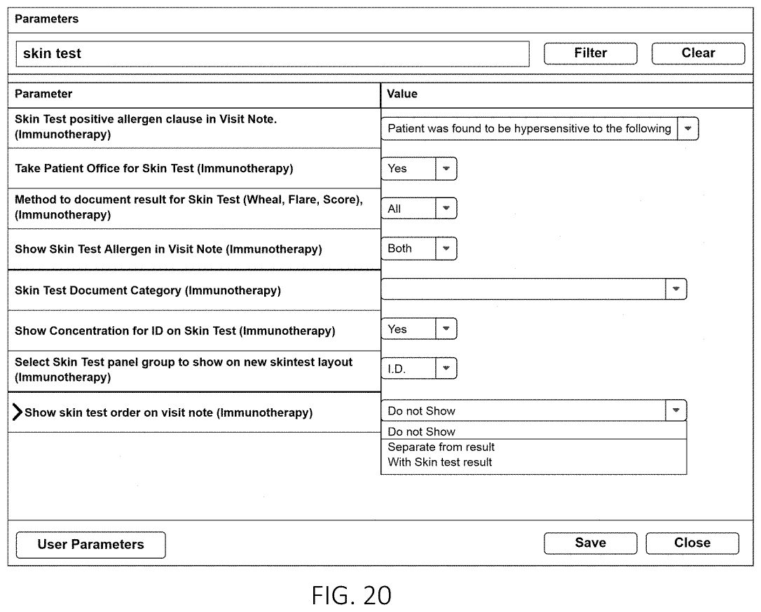

At step 1901, the central system module 1200 automatically updates the skin test details. One set of exemplary parameters depicted on the user interface for updating the skin test details are shown in FIG. 20.

Since the central system module 1200 also manages treatment of allergy, the central system module 1200 records all steps and progression taken in allergy treatment. As such, referring back to FIG. 14, all allergy steps are recorded and updated at Step 1417 to the server, IMS, or an electronic health record.

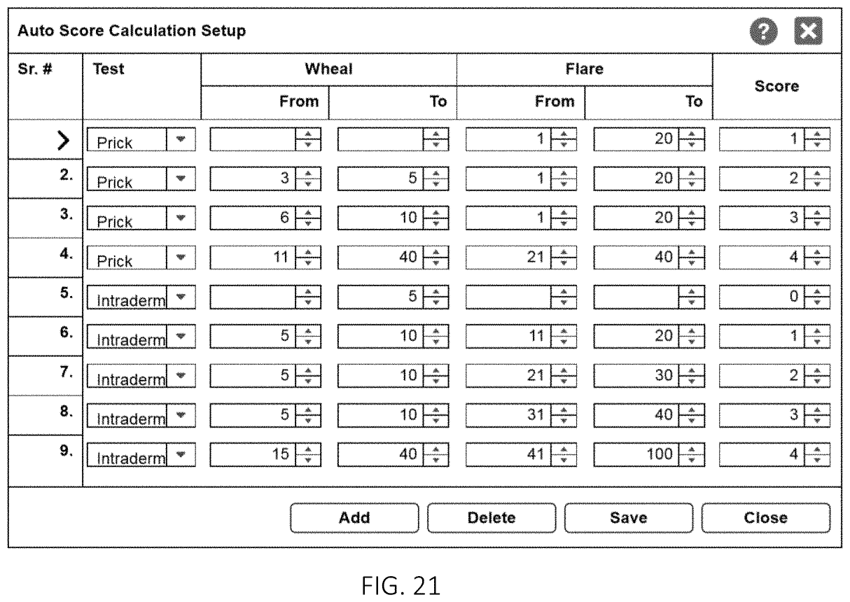

The user interface that is generated by the central system module 1200 is one of the tools for navigating through decision logic and complex decision trees used in analyzing a reaction and performing all the steps in testing for allergies and managing allergy treatment. Although several examples of the user interface have been provided in the application, a few additional screens of the user interface are depicted below in FIGS. 21-25. These are by no way limited and additional screens that relate to performing the steps and functions listed in the application are also contemplated. FIGS. 21-25 are exemplary and in no particular order of rendition on the user interface. They may be provided upon instruction of the central system module 1200 based on the stage of the process or if a certain input from the user is needed or preferred.

FIG. 21 depicts one exemplary screen of the user interface that shows wheal flare value determination according to the disclosed embodiments. The central system module 1200 causes the screen to appear and allows the user to set predetermined values for wheals and flares. Accordingly, the user will receive positive and negative allergy results for allergen based on these entered values.

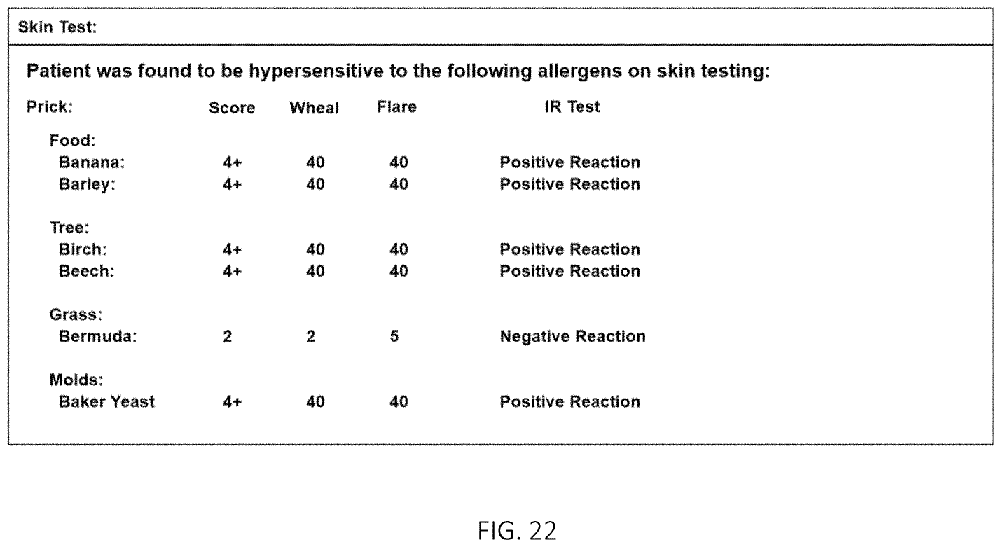

FIG. 22 depicts one exemplary screen of the user interface that shows skin test order and its parameters according to the disclosed embodiments. As shown in FIG. 22, allergens relating to food, trees, grass, and molds are depicted along with their associated score and measurements of wheal and flare. In this particular embodiment, the user interface is produced when the user of the system selects “Do Not Show” selection from the show skin test order. The screen reflects which allergens were found to be hypersensitive for the patient.

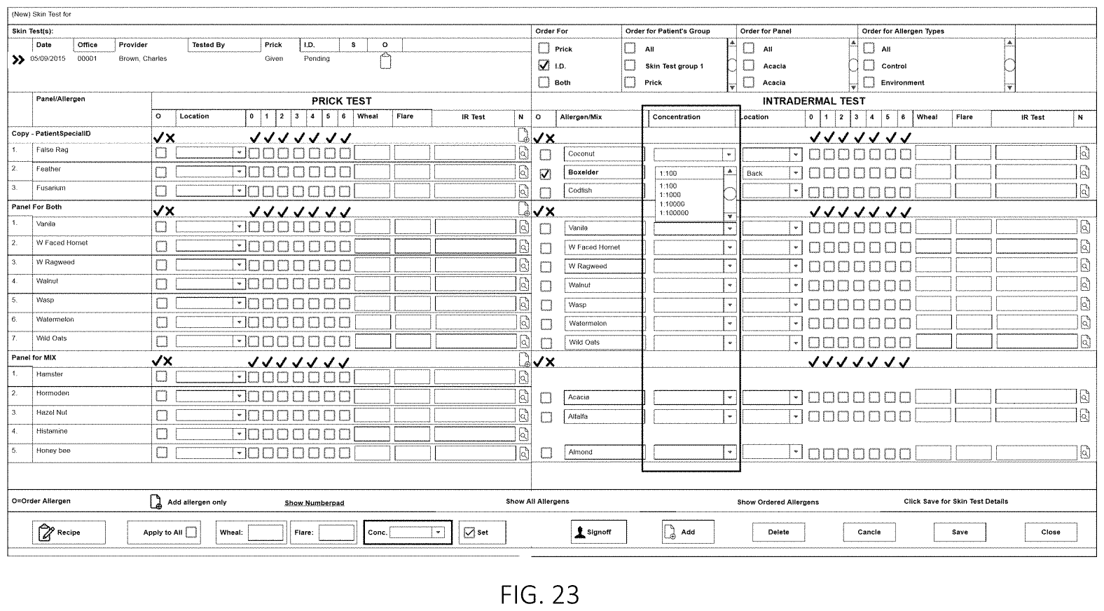

FIG. 23 depicts one exemplary screen of the user interface that shows concentration columns for an intradermal test according to the disclosed embodiments. The central system module 1200 causes the screen to appear and allows the user to select from a set of concentration values for each of the intradermal tests to be performed. For example, as shown in FIG. 23, the user is attempting to select a concentration value for a coconut allergen. The concentration value ranges shown are 1:100, 1:1000, 1:10000, and 1:100000. Although the values ranging from 100 to 100,000 are shown, other concentration values are also contemplated. The concentration levels are used in preparation of vaccines. When the concentration features for intradermal tests are enabled, the user can also set a default concentration for the vials.

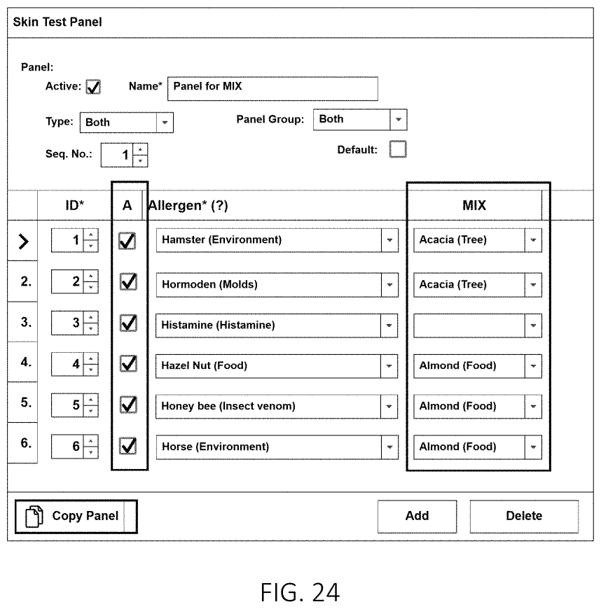

FIG. 24 depicts one exemplary screen of the user interface that shows a skin panel creation according to the disclosed embodiments. As discussed in FIGS. 8b, 8c, and 9b, a skin allergy test consists of a variety of panels or grouping of allergens. The skilled operator can use a specific quadrant (or more) and draw a grid accordingly on the patient's back (e.g. FIG. 6a) on which the allergens for that panel will be injected. In order to keep track of which allergens were injected in which part of the grid drawn in FIG. 6a, the skilled operator can use the skin panel creation screen produced by the central system module 1200 to create a panel and manage the panel while injecting the associated allergens into the patient. For example, the skilled operator can keep track of Allergen 1, which is an Allergen for Hamster environment with a mix of an Acacia Tree being injected in section one of the grid while Homoden allergen with a mix of acacia tree being injected in section 2 of the grid. The user interface with the help of this screen thus provides for recording allergen location in the grid as well as using it as planning for creating allergen panels.

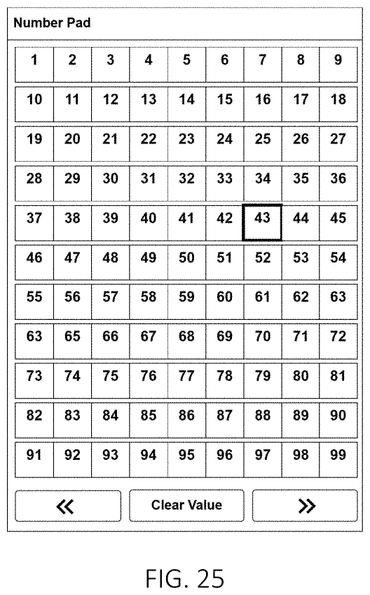

FIG. 25 depicts one exemplary screen of the user interface that shows a numeric keypad for entering wheal and flare results according to the disclosed embodiments. Once the panel of allergens is administered to the patient, the results can be recorded by selecting the location of each allergen on the grid created, such as in FIG. 6a, but selecting a number and inputting the reaction. As shown in FIG. 25, the skilled operator has selected location 43 to input wheal and flare measurements.

FIG. 26 illustrates a block diagram depicting one start-to-finish cycle of performing a skin test and managing end-to-end allergy treatment, scheduling, and billing according to the disclosed embodiments. As depicted in FIG. 26, yet another embodiment of a step by step cycle from start-to-end is provided. In this embodiment, patient scheduling, billing, and visitation notes are also included. Based on a patient visit, the central system module 1200 causes automatic generation of patient visitation notes. These notes are analogues to a diagnosis or patient chart that is typically prepared by a physician. It may also be an update that records patient treatment during a doctor's office or allergy clinic visit. Scheduling and billing associated to the patient visit is also produced by central system module 1200. It will be apparent to those skilled in the art that various modifications and variations can be made in the smart bottle assembly and associated methods without departing from the spirit or scope of the invention. Thus, it is intended that the present invention covers the modifications and variations of the embodiments disclosed above provided that the modifications and variations come within the scope of any claims and their equivalents.

What is claimed is:

1. A computer implemented and automated method for detecting and measuring an allergic reaction using an electronic device and electronically monitoring and managing allergy treatment comprising:
accessing, by a server computer, an infrared electronic module, wherein the infrared electronic module includes a body, a handle, an infrared temperature sensor connected to a microprocessor, and a trigger button to operate the infrared electronic module; wherein the server computer includes a server processor, server memory, and a server network interface;
the server processor activating the infrared electronic module, wherein such activation results in the infrared electronic module obtaining a temperature reading of a wheal formed on the patient's body, wherein the wheal is formed as a form of reaction in response to an allergen injected into the patient's body;
the server processor receiving the temperature reading of the wheal, wherein receiving comprises wirelessly connecting the server processor with the infrared electronic module and transmitting the temperature reading of the wheal from the infrared electronic module to the server processor;
storing the temperature reading of the wheal in the server memory;
analyzing the temperature reading of the wheal;
accessing, by the server processor, a camera, wherein the camera includes a lens for capturing an image;
the server processor activating the camera, wherein such activation results in the camera obtaining an image of the wheal whose temperature was obtained by the infrared electronic module, wherein the image capture includes focusing the lens of the camera on the wheal such that the entire wheal along with its dimensions are captured in the image;
the server processor receiving the captured image from the camera, wherein receiving comprises wirelessly connecting the server processor with the camera and transmitting the image of the wheal along with its dimensions from the camera to the server processor;
storing the image of the wheal along with its dimensions in the server memory;
the server processor analyzing the image of the wheal and its dimensions;
the server processor scoring the allergen injected into the patient's body that caused the formation of the wheal to determine if a positive or negative reaction has occurred, wherein the scoring is reflective of the temperature reading and the wheal image and dimensions; and
the server processor causing the display of the scoring results to be depicted on a user interactable user interface.

2. The computer implemented and automated method of claim 1, further comprising the server comparing the temperature reading of the wheal obtained by the infrared electronic module with the image of the wheal along with its dimensions obtained by the camera.

3. The computer implemented and automated method of claim 2, wherein comparing includes determining a range of measurement that are within a predetermined range for a temperature reading obtained by the infrared electronic module.

4. The computer implemented and automated method of claim 2, wherein comparing includes determining a temperature range that are within a predetermined range for a wheal dimension obtained by the camera.

5. The computer implemented and automated method of claim 2, wherein the server producing an error message if either the temperature reading is not within a predetermined range computer based on the wheal dimensions or if the wheal dimensions are not in a predetermined range computed based on the temperature reading.

6. The computer implemented and automated method of claim 1, wherein the infrared electronic module is a handheld device having an optical lens for reading radiated waves that emanate from the wheal.

7. The computer implemented and automated method of claim 1, wherein the camera accessed by the server processor is an array of camera sub-modules, wherein each camera sub-module is housed in a flexible, bendable, and twistable prong, and each camera sub-module is capable of taking an image of a specific wheal that is formed on the patient's body.

8. The computer implemented and automated method of claim 7, wherein each camera sub-module can be flexed, bended, and or twisted to aim at a specific area of a patient's body where an allergen is injected.

9. The computer implemented and automated method of claim 7, wherein the server computer is capable of instantly computing the images taken by all the camera sub-modules.

10. The computer implemented and automated method of claim 1, further comprising performing an image enhancement on the image that the server processor received from the camera, the image enhancement comprising the steps of:
analyzing image clarity;
analyzing image completeness;
analyzing image color;
analyzing the pixels of image; and
displaying results of the analysis on the user interactable user interface.

11. The computer implemented and automated method of claim 10, wherein analyzing the image clarity, completeness, and color, includes
comparing the clarity, completeness, and color to a predetermined setting;
displaying an error message if the clarity, completeness, and color do not match the predetermined setting; and
activating the camera to obtain a second image of the wheal.

12. The computer implemented and automated method of claim 11, wherein the predetermined setting is a value that is computed based on the temperature readings taken by the infrared electronic module.

13. The computer implemented and automated method of claim 10, wherein analyzing the image clarity, completeness, and color, includes applying a digital image processing method, such as the Markov method, to determine image accuracy.

14. The computer implemented and automated method of claim 1, wherein scoring further comprises, the server processor producing a score between 0 and 6, wherein the numbers represent a sliding scale, wherein a 0 represents a negative reaction and a 6 represents a positive reaction.

15. The computer implemented and automated method of claim 1, further comprising a guidance module for guiding an operator of the server computer, the server processor utilizing the guidance module by performing the steps comprising:
communicating with the guidance module to present an interactive display on the user interactable user interface;
the server processor of the server computer recommending one or more allergens that are to be injected into the patient's body and presenting the recommendation on the user interactable user interface;
the server processor of the server computer producing an image that represents the back of the patient's body on the user interactable user interface, wherein the image depicts a grid of markers;
the server processor of the server computer associating each allergen to be injected with one specific marker from the grid; and
the server processor of the server computer activating a testing timer for each allergen, wherein the timer represents the amount of time the allergen is to remain in the patient's body for testing.

16. The computer implemented and automated method of claim 15, further comprising allowing the operator of the server computer to accept or reject the recommendations of allergens and adjust the testing timer to a desired time.

17. The computer implemented and automated method of claim 1, further comprising a vaccination preparation module of the server computer, the server processor utilizing the vaccination preparation module by performing the steps comprising:
communicating with the vaccination preparation module to present an interactive display on the user interactable user interface;
the server processor of the server computer recommending an antigen to be used, wherein the antigen recommended is determined to produce a positive allergic reaction to the patient;
the server processor of the server computer receiving inputs for preparing a vaccination, wherein the inputs are provided by a user through a keyboard or through the interactable user interface associated with the server computer;
the server processor of the server computer analyzing the inputs received and the antigens recommended to present a recommended vaccination, wherein the server processor's a recommendation of the vaccination includes quantities of antigen to be used; and
the server processor displaying a prompt on the user interactable interface thereby allowing a user of the user interactable interface to select a concentration of vaccination that is to be placed in a vaccination vial.

18. The computer implemented and automated method of claim 17, further comprising a vaccination scheduling module, wherein the server processor activates the vaccination scheduling module to perform the steps comprising:
the server processor receiving an input for scheduling a vaccination;
the server processor calendaring the vaccination and updating the vaccination schedule for a facility based on the input received; and
the server processor documenting the vaccination and updating the patient's records in a database associated with the server computer, wherein such updating includes updating the patient's skin test results based on the taking of the vaccination on the scheduled date.

19. The computer implemented and automated method of claim 1, further comprising:
the server processor accessing the server memory to determine if prior allergy test results for the patient exist in the server memory;
providing an option to the user to add prior allergy test results if they exist in the server memory; and
the server processor receiving data from a prick and skin tests and adding the results of the test to the server memory.

20. The computer implemented and automated method of claim 1, wherein the server processor further causing a skin test summary to be displayed on the user interactable interface, wherein the skin test summary includes a list of locations on the patient's body where the allergens were injected, a wheal dimension for each location, a flare dimension for each location, a score for each location, a temperature reading for each location, and a reaction associated with each location.
